(19) United States
(12) Patent Application Publication
(10) Pub. No.: US 2025/0179502 A1
(43) Pub. Date: Jun. 5, 2025

(54) TARGETING NEUROPILIN 2 (NRP2) IN LETHAL PROSTATE CANCER

(71) Applicant: UNIVERSITY OF MASSACHUSETTS, Boston, MA (US)

(72) Inventors: Arthur M. MERCURIO, Boston, MA (US); Mengdie WANG, Holden, MA (US)

(21) Appl. No.: 18/841,903

(22) PCT Filed: Feb. 27, 2023
(86) PCT No.: PCT/US2023/063339

§ 371 Date: Aug. 27, 2024

Related U.S. Application Data
(60) Provisional application No. 63/314,846, filed on Feb. 28, 2022.

(51) Int. Cl. C12N 15/113; A61K 31/337; A61K 31/36; A61K 31/404; A61K 31/473; A61K 31/555; A61K 33/243; A61K 38/12; A61K 38/17; A61K 45/06; A61P 35/04; C07K 16/28; G01N 33/573; G01N 33/574

(52) U.S. Cl. CPC C12N 15/1138; A61K 31/337; A61K 31/36; A61K 31/404; A61K 31/473; A61K 31/555; A61K 33/243; A61K 38/12; A61K 38/179; A61K 45/06; A61P 35/04; C07K 16/2863; G01N 33/573; G01N 33/57434; G01N 33/57488; G01N 33/57492; C07K 2317/24; C07K 2317/52; C07K 2317/565; C12N 2310/14; C12N 2310/531; G01N 2333/46; G01N 2333/70596; G01N 2333/988; G01N 2474/20

(57) ABSTRACT

Described herein are compositions and methods for use in targeting neuropilin 2 (NRP2) in lethal prostate cancer, e.g., in metastatic castration-resistant prostate cancer (mCRPC) or neuroendocrine prostate cancer (NEPC).

### CLAIM OF PRIORITY

This application claims the benefit of U.S. Provisional Patent Application Ser. No. 63/314,846, filed on Feb. 28, 2022. The entire contents of the foregoing are hereby incorporated by reference.

### FEDERALLY SPONSORED RESEARCH OR DEVELOPMENT

This invention was made with Government support under Grant Nos. CA168464 and CA218085 awarded by the National Institutes of Health and Grant No. W81XWH2110123 awarded by the Department of Defense. The Government has certain rights in the invention.

### TECHNICAL FIELD

Described herein are compositions and methods for use in targeting neuropilin 2 (NRP2) in lethal prostate cancer, e.g., in metastatic castration-resistant prostate cancer (mCRPC) or neuroendocrine prostate cancer (NEPC).

### BACKGROUND

Aggressive prostate carcinoma (PCa) with metastasis is a lethal disease that consists multiple subtypes with significant heterogeneity that is primarily treated with androgen deprivation therapy (ADT) ¹. While initially effective, primary ADT inevitably leads to resistance, a lethal form of the disease termed metastatic castration-resistant prostate cancer (mCRPC) ^{2,3}. A subset of mCRPC can evolve into neuroendocrine prostate cancer (NEPC) by upregulation of regulators that drive stemness and lineage plasticity in response to pressure from therapies ⁴. It is an aggressive form of PCa that is associated with rapid progression, drug resistance and a very poor prognosis with most patients survive less than 1 year^{5,6}. Currently, there are no established therapies for treating NEPC 7.

### SUMMARY

Provided herein are methods of treating a subject (e.g., a human subject) who has metastatic castration-resistant prostate cancer (mCRPC) or neuroendocrine prostate cancer (NEPC). The methods include administering to the subject a therapeutically effective amount of an inhibitor of Neuropilin 2 (NRP2). Also provided are inhibitors of Neuropilin 2 (NRP2) for use in a method of treating a subject who has metastatic castration-resistant prostate cancer (mCRPC) or neuroendocrine prostate cancer (NEPC), or for use in the preparation of a medicament for treating a neuroendocrine prostate cancer (NEPC) or mCRPC. In some embodiments, the inhibitor is selected from the group consisting of antibodies or antigen binding fragments thereof: inhibitory peptides: small molecules; and inhibitory nucleic acids.

In some embodiments, the antibody is an antibody or antigen-binding fragment thereof that specifically binds to a human neuropilin-2 (NRP2), e.g., as described in U.S. Pat. No. 11,505,610, or an antibody or antigen-binding fragment thereof of antibody aNRP2-28 (e.g., v2 or v4, see US20210163606) or aNRP2-10v10 (see US20210163606). In some embodiments, inhibitory peptide comprises soluble NRP2 B domain, optionally with mutations R287E and N290D. In some embodiments, the small molecule is Zafirlukast, Actinomycin D, Dihydrexidine, or a benzamidine inhibitor of VEGF-C binding to NRP2. In some embodiments, the inhibitory nucleic acids are antisense oligonucleotides (ASOs) or single- or double-stranded RNA interference (RNAi) compounds such as siRNA or shRNA that bind to a nucleic acid encoding NRP2.

In some embodiments, the methods further include administering a treatment comprising an anti-angiogenic agent, e.g., a VEGF inhibitor, e.g., an antibody (e.g., bevacizumab, ramucirumab, or ranibizumab); aflibercept: or a Tyrosine kinase inhibitor (TKI) (e.g. sunitinib, pazopanib, sorafenib, nilotinib, axitinib, or dasatinib).

Also provided herein are methods for treating a neuroendocrine prostate cancer (NEPC) or or mCRPC in a subject, e.g., a human subject, in need thereof, comprising administering to the subject an antibody or an antigen-binding fragment thereof that specifically binds to a human neuropilin-2 (NRP2) polypeptide at an epitope in the neuropilin b1 domain of NRP2, thereby treating the NEPC or mCRPC in the subject in need thereof. Also provided are pharmaceutical compositions as described herein for use in treating a neuroendocrine prostate cancer (NEPC) or mCRPC in a subject in need thereof, e.g., comprising an antibody or an antigen-binding fragment thereof that specifically binds to a human neuropilin-2 (NRP2) polypeptide, e.g., at an epitope in the neuropilin b1 domain of NRP2. Further. provided is the use of a pharmaceutical composition as described herein in the preparation of a medicament for treating a neuroendocrine prostate cancer (NEPC) or mCRPC in a subject in need thereof, optionally wherein the composition includes an antibody or an antigen-binding fragment thereof, that specifically binds to a human neuropilin-2 (NRP2) polypeptide, e.g., at an epitope in the neuropilin b1 domain of NRP2.

In some embodiments, the antibody or antigen-binding fragment thereof comprises: a heavy chain variable region (V_{H}) sequence that comprises complementary determining region (CDR) V_{H}CDR1, V_{H}CDR2, and V_{H}CDR3 sequences set forth in SEQ ID NOs: 1-3, respectively, and a light chain variable region (V_{L}) sequence that comprises complementary determining region V_{L}CDR1, V_{L}CDR2, and V_{L}CDR3 sequences set forth in SEQ ID NOs: 4-6, respectively: or a V_{H} sequence that comprises V_{H}CDR1, V_{H}CDR2, and V_{H}CDR3 sequences set forth in SEQ ID NOs: 7-9, respectively, and V_{L} sequence that comprises V_{L}CDR1, V_{L}CDR2, and V_{L}CDR3 sequences set forth in SEQ ID NOs: 10-12, respectively. In some embodiments, the V_{H} sequence comprises a sequence at least 80, 85, 90, 95, 97, 98, 99, or 100% identical to SEQ ID NO: 13, and the V_{L} sequence comprises a sequence at least 80, 85, 90, 95, 97, 98, 99, or 100% identical to SEQ ID NO: 14: or the V_{H} sequence comprises a sequence at least 80, 85, 90, 95, 97, 98, 99, or 100% identical to SEQ ID NO: 15, and the V_{L} sequence comprises a sequence at least 80, 85, 90, 95, 97, 98, 99, or 100% identical to SEQ ID NO: 16: or the V_{H} sequence comprises a sequence at least 80, 85, 90, 95, 97, 98, 99, or 100% identical to SEQ ID NO: 17, and the V_{L} sequence comprises a sequence at least 80, 85, 90, 95, 97, 98, 99, or 100% identical to SEQ ID NO: 18. See U.S. Pat. No. 11,505,610 and US20210163606.

In some embodiments, the antibody or antigen-binding fragment thereof is a humanized monoclonal antibody that comprises a human IgG4 Fc domain. See, e.g., U.S. Pat. No. 11,505,610 and US20210163606.

In some embodiments, the subject has NEPC. In some embodiments, the NEPC is characterized by cell morphology/histology and/or expression of one or more NEPC markers, e.g., synaptophysin, chromogranin A (CgA), neuron-specific enolase (NSE), and/or CD56.

In some embodiments, the methods include determining the presence or absence of an NEPC in a tissue sample from the subject; and administering the antibody or antigen-binding fragment thereof to the subject if the NEPC is present in the tissue sample. In some embodiments, the methods include determining the presence or absence of the NEPC in the tissue sample by cell morphology/histology; and administering the antibody or antigen-binding fragment thereof to the subject if the morphology/histology indicates the presence of the NEPC in the tissue sample. In some embodiments, the methods include determining the presence or absence of the NEPC in the tissue sample by immunohistochemistry (IHC) for one or more NEPC markers; and administering the antibody or antigen-binding fragment thereof to the subject if the IHC is positive for the one or more NEPC markers (e.g., synaptophysin, chromogranin A (CgA), neuron-specific enolase (NSE), and/or CD56).

In some embodiments, the methods include obtaining the tissue sample from the subject; the tissue sample can be, e.g., a liquid biopsy optionally a blood sample, a surgical sample, or other biopsy sample obtained from the subject, optionally a biopsy of prostate cancer tissue.

In some embodiments, the human subject has received at least 1 or 2 lines of systemic therapy for prostate cancer and has relapsed from the last systemic therapy, e.g., optionally wherein the last systemic therapy is selected from one or more of hormonal therapy via surgical or chemical castration (LHRH agonist), chemotherapy, and radiopharmaceutical therapy.

In some embodiments, the methods include administering the inhibitor of NRP2, e.g., antibody or antigen-binding fragment thereof, in combination with a treatment comprising an anti-angiogenic agent, or at least one or two additional chemotherapeutic agents. In some embodiments, the methods include the at least one additional chemotherapeutic agent is selected from etoposide, carboplatin, cisplatin, and docetaxel. In some embodiments, the at least two additional chemotherapeutic agents are selected from etoposide+carboplatin, etoposide+cisplatin, and docetaxel +carboplatin. In some embodiments, the anti-angiogenic agent is a VEGF inhibitor, e.g., an antibody (e.g., bevacizumab, ramucirumab, or ranibizumab); aflibercept; or a Tyrosine kinase inhibitor (TKI) (e.g., sunitinib, pazopanib, sorafenib, nilotinib, axitinib, or dasatinib).

Additionally, provided herein are kits, e.g., patient care kits, that include means for determining the presence or absence of a neuroendocrine prostate cancer (NEPC) in a tissue sample from a human patient with prostate cancer (reagents for performing immunohistochemistry (IHC) on one or more NEPC markers); and an antibody or an antigen-binding fragment thereof that specifically binds to a human neuropilin-2 (NRP2) polypeptide, e.g., at an epitope in the neuropilin b1 domain of NRP2, as described herein. In some embodiments, the one or more NEPC markers are selected from synaptophysin, chromogranin A (CgA), neuron-specific enolase (NSE), and CD56.

In some embodiments, the tissue sample is a liquid biopsy optionally a blood sample, a surgical sample, or other biopsy sample obtained from the patient, optionally a biopsy of prostate cancer tissue.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-E. NRP2 correlates with PD-L1 expression in prostate cancer cells.

(A) Percentage of NRP2 and VEGF-A gene alterations in PCa patients based on CbioPortal.

(B) The strategy used to sort PC3 cells into NRP2^{High} and NRP2^{LOW} populations using flow cytometry.

(C) Gene set enrichment analysis (GSEA) data shows pathways that were significantly enriched in NRP2^{High} comparing to NRP21.0″ population.

(D) Heat-map shows all the genes that were significantly altered in NRP2^{High} comparing to NRP2^{Low} population.

(E) Bar graphs show fold change of NRP2/18S and PD-L1/18S mRNA in NRP2^{High} comparing to NRP2^{Low} population from 2 different PCa cell lines with 3 independent experiments by qPCR.

FIGS. 2A-I. VEGF/NRP2 signaling contributes to PD-L1 expression in prostate cancer cells.

(A) Bar graphs show fold change of NRP2/18S and PD-L1/18S mRNA of control cells (PC3-WT or PC3-sCtrl) versus cells with NRP2 deletion (PC3NRP2KO, PC3-shNRP2-1 or PC3-shNRP2-2) from 3 independent experiments determined by qPCR.

(B) NRP2 expression was depleted in PC3 cells using CRISPR/Cas9. NRP2 and PD-L1 protein levels were compared to control (WT) cells by immunoblotting, a-Tubulin was used as loading control.

(C) Flow cytometry of cell surface PD-L1 in PC3^{WT} and PC3^{NRP1KO} cells.

(D) Representative images of PC3^{WT} cells and PC3NRP2KO cells stained with PD-L1 and Hoechst 33342 (nuclei). Scale bar: 10 μm.

(E) Flow cytometry of cell surface PD-L1 in PC3-siCtrl cells, siVEGF-A cells, and siVEGF-C cells.

(F) Bar graphs show fold change of NRP2/18S and PD-L1/18S mRNA in PC3-shCtrl cells versus PC3-shNRP2, and PC3-shNRP2-NRP2-GFP cells from 3 independent experiments determined by qPCR.

(G) Flow cytometry of cell surface PD-L1 in PC3-shCtrl, PC3-shNRP2, and PC3-shNRP2-NRP2-GFP cells.

(H) Flow cytometry of NRP2 in TRAMP-C2-shGFP, TRAMP-C2-shNRP2-1, and TRAMP-C2-shNRP2-2 cells.

(1) Flow cytometry of PD-L1 in TRAMP-C2-shGFP, TRAMP-C2-shNRP2-1, and TRAMP-C2-shNRP2-2 cells.

FIGS. 3A-F. NRP2 promotes PD-L1 transcription through activation of Hippo pathway.

(A) Bar graphs show fold change of CTGF/18S and CYR61/18S mRNA in PC3-shCtrl versus PC3-shNRP2 from 3 independent experiments determined by qPCR.

(B) Representative images of PC3^{WT} cells and PC3NRP2KO cells stained with YAP and Hoechst 33342 (nuclei). Scale bar: 10 μm. Single channel images of YAP only are shown on the right. Bar graph shows quantification of YAP localization in the nuclei, cytoplasm, or both. Data was analyzed from 3 independent experiments.

(C) Representative images of PC3^{WT} cells and PC3^{NRP1KO} cells stained with TAZ and Hoechst 33342 (nuclei). Scale bar: 10 μm. Single channel images of TAZ only are shown on the right. Bar graph shows quantification of YAP localization in the nuclei, cytoplasm, or both. Data was analyzed from 3 independent experiments.

(D) Bar graphs show fold change of PD-L1/18S mRNA in PC3 treated with DMSO versus PC3 treated with YAP inhibitor (Verteporfin) from 3 independent experiments determined by qPCR.

(E) Bar graphs show fold change of PD-L1/18S mRNA in PC3^{WT} versus PC3NRP2KO, and PC3NRP2KO-TAZ (4SA) cells from 3 independent experiments determined by qPCR.

(F) (top left) Flow cytometry of PD-L1 in PC3^{NRP1KO} and PC3^{NRP2KO} TAZ (4SA) cells, (top right) Flow cytometry of PD-L1 in PC3-shNRP2-1, and PC3-shNRP2-1-TAZ (4SA) cells, (bottom) Flow cytometry of PD-L1 in PC3-shNRP2-2, and PC3-shNRP2-2-TAZ (4SA) cells.

FIGS. 4A-D. NRP2 inhibits T cell activation in vitro.

(A) Schematic shows the co-culture experiment of tumor cells with PBMCs pre-labeled with proliferation dye and activated with anti-CD3/CD28 beads. Cells were analyzed by flow and gated for live cells using live/dead kit. Live cells were gated for CD45⁺, followed by CD3⁺ to mark for T cells. T cells were then gated for CD8⁺ or CD4⁺T cells.

(B) Representative flow images show the percentage of proliferating CD4⁺ (top) or CD8⁺(bottom) in PBMCs without co-culture with tumor cells or activation beads (left), PBMCs co-cultured with PC3-shCtrl and activation beads (middle left), PBMCs co-cultured with PC3-shNRP2 and activation beads (middle right), and PBMCs co-cultured and activation beads only as positive control (right).

(C) Quantification of the percentage of proliferating CD4⁺T cells (top) or CD8⁺T cells (bottom) in PBMCs co-cultured with different ratio of PC3-shCtrl, PC3-shNRP2-1, or PC3-shNRP2-2 cells. Statistical significance determined by multiple comparisons 2way ANOVA (*, 0.05>P≥0.01, *** 0.001>P≥0.0001, **** 0.0001>P).

(D) Quantification of IFN-g, IL-17A, and IL-2 in the supernatant from PBMCs co-cultured with PCa cells under different condition.

FIGS. 5A-D. NRP2 loss in human PCa cells results in immune mediated tumor elimination in vivo.

(A) Schematic shows the establish of humanized mouse model.

(B) Quantification of tumor volume in mice injected with PC3-shCtrl cells with or without engrafting of human PBMCs. PBMCs from two different doners were used. Arrowhead indicates the date of PBMCs engraftment.

(C) Quantification of tumor volume in mice injected with PC3-shNRP2 cells with or without engrafting of human PBMCs. PBMCs from two different doners were used. Arrowhead indicates the date of PBMCs engraftment.

(D) The percentage of tumor free mice among mice injected with PC3-shCtrl or PC3-shNRP2 cells on day 40 after PBMC injection.

FIGS. 6A-E. Therapeutic targeting NRP2 results in significant tumor regression in a syngeneic NEPC mouse model in vivo.

(A) Flow cytometry of PD-L1 in TRAMP-C2 cells treated with IgG control or the aNRP2-28 Ab.

(B) Schematic shows the treatments in the C57BL/6J mice syngeneic mouse model or NSG model.

(C) Quantification of tumor volume in C57BL/6J mice syngeneic mouse model (top) or NSG model (bottom) injected with TRAMP-C2 cells and treated with either IgG control or the aNRP2-28 Ab. Arrowhead indicates the starting date of treatment.

(D) Immunofluorescent images of tissue samples stained with PD-L1 and Hoechst 33342 (nuclei) from TRAMP-C2 tumors treated with either IgG or the aNRP2-28 Ab. Scale bar: 100 μm.

(E) Quantification of PD-L1 fluorescent intensity in tissue samples from TRAMP-C2 tumors in C57BL/6J mice treated with IgG or the aNRP2-28 Ab antibody.

FIGS. 7A-B. Therapeutic targeting NRP2 results in a significant increase of tumor infiltrating immune cells.

(A) Quantification of tumor-infiltrating immune cells in TRAMP-C2 tumors treated with IgG control (n=6 mice), aNRP2 (aNRP2-28) antibody (n=6 mice), or aPD-L1 antibody (n=5 mice) using flow cytometry. The following cells were analyzed: Lymphocytes (CD45+), T cells (CD45+CD3+), CD4⁺T cells (CD45+CD3+CD4⁺), CD8⁺T cells (CD45+CD3+CD8⁺), NK cells (CD45+CD3-NKp46+), activated T cells (CD45+CD3+CD69+), activated CD8⁺T cells (CD45+CD3+CD8⁺Granzyme B+), and activated NK cells (CD45+CD3-NKp46+Granzyme B+). Data shown as means±SD. Statistical significance was determined by two-sided, unpaired t test. * P<0.05, ** P<0.01. *** p<0.001, **** p<0.0001.

(B) Immunofluorescent images of tissue samples stained with CD3 (left), CD8 (middle), Granzyme B (right) and Hoechst 33342 (DNA) from TRAMP-C2 tumors in C57BL/6J mice treated with either IgG (top) or the aNRP2-28 Ab (bottom). Scale bar: 100 μm.

FIGS. 8A-F. NRP2/VEGF signaling contributes to PD-L1 expression.

(A). Schematic shows the design to use CRISPR/Cas9 to generate NRP2 knockout cells.

(B) PCR results show homozygous knockout of exon-2 in NRP2 gene in the PC3^{NRP2KO} cells.

(C) Bar graphs show fold change of NRP2/18S mRNA in PC3^{NRP1KO} or PC3-shNRP2 versus control cells (PC3^{WT} or PC3-shCtrl) from 3 independent experiments determined by qPCR.

(D) Bar graphs show fold change of VEGF-A/18S mRNA in PC3-siCtrl versus PC3-si-VEGF-A cells from 3 independent experiments determined by qPCR.

(E) Flow cytometry of GFP in PC3-shCtrl, PC3-shNRP2 and PC3-shNRP2-NRP2-GFP cells.

(F) Western blots show PD-L1 expression in PC3-shNRP2 cells and PC3-shNRP2-PD-L1-EGFP cells. Actin was used as loading control.

FIG. 9. NRP2 inhibits T cell viability through PD-L1.

Quantification of % of live T cells in PBMCs co-cultured with different ratio of PC3-shCtrl, PC3-shNRP2-1, PC3-shNRP2-2, PC3-shNRP2-NRP2-GFP, or PC3-shNRP2-PD-L1-EGFP cells.

FIGS. 10A-D. Humanized mouse model exgrafted with aggressive PC cells show tumor infiltrating immune cells.

(A) % of Tumor free mice in PC3-shCtrl and PC3-shNRP2 cells following days after tumor cells injection.

(B) Quantification of % of CD45⁺, CD3⁺, CD4⁺, and CD8⁺ cells in peripheral blood 2 weeks after injection with hPBMCs from mice injected with PC3-shCtrl cells or PC3-shNRP2 cells.

(C) Quantification of % of CD45⁺, CD3⁺, CD4⁺, and CD8⁺ cells in peripheral blood 4 weeks after injection with hPBMCs from mice injected with PC3-shCtrl cells or PC3-shNRP2 cells.

(D) Quantification of % of CD45⁺, CD3⁺, CD4⁺, and CD8⁺ cells in tumors 40 days after injection with hPBMCs from mice injected with PC3-shCtrl cells.

FIGS. 11A-C. Therapeutic inhibition of VEGF binding to NRP2 activates anti-tumor immunity in a syngeneic model of prostate cancer that is resistant to aPD-L1 therapy.

(A) Shown is quantification of tumor volume in C57BL/6J mice injected with TRAMP-C2 cells and treated with either IgG control or aPD-L1 antibody. Error bars represent SEM. n=8 mice for each group.
(B) Shown are representative histological images of whole tissue scans of TRAMP-C2 tumors treated with either IgG control (left), aNRP2 antibody (middle), or aPD-L1 antibody (right) in C57BL/6J mice. Tumors were stained with H&E and show extensive tumor necrosis in the aNRP2 treated group. Scale bar, 1 mm.

(C) Shown is the quantification of the percentage of necrotic region in tumor sections from TRAMP-C2 tumors treated with IgG control or aPD-L1 antibody in C57BL/6J (n=10 for IgG group and 8 for aPD-L1 group). Error bars represent SEM. Statistical significance was determined by two-sided, unpaired t test. ns, not significant.

FIGS. 12A-G. Therapeutic inhibition of NRP2/VEGF interactions sensitizes NEPC organoids to immune cell-mediated killing.

(A) Shown is the percentage of samples with NRP2, VEGF-A, and VEGF-C gene amplification isolated from patients with prostate cancer. Data were obtained from cBioPortal. Data from patients with NEPC were derived from a multi-institute neuroendocrine prostate cancer study^{67.}

(B) Shown is the correlation between NRP2 and CD274 mRNA expression in patients with metastatic prostate cancer based SU2C/PCF Dream Team, Abida et al., 201962 datasets on cBioPortal.

(C) AR and NEPC scores were quantified in NRP2^{high} PD-L1high and NRP2^{low} PD-L1^{low} population (determined by 80th and 20th percentile of NRP2 and CD274 expression) in patient data from a SU2C/PCF Dream Team study⁶². AR and NEPC scores were reported in this study. Statistical significance was determined by two-sided, unpaired t test, ** P<0.01, **** P<0.0001.

(D) Representative images show OWCM1262 NEPC organoids stained with NRP2 or PD-L1. DNA is stained with Hoechst. Scale bar: 10 μm.

(E) OWCM1262 NEPC organoids were treated with either control IgG or aNRP2 (aNRP2-10) for 5 days and PD-L1 surface expression was quantified by flow cytometry. Statistical significance was determined by two-sided, unpaired t test (n=5 biological replicates). ** P<0.01.

(F) OWCM1262 NEPC organoids were treated with either IgG control or aNRP2 (aNRP2-10) and were co-cultured in the presence or absence of PBMCs that had been activated using aCD3/CD28 beads. Flow cytometry show percentage of live/dead tumor cells after 5 days.

(G) Shown is the quantification of percentage of dead tumor cells shown in (F). Data are shown as means+SD. Statistical significance was determined by two-sided, unpaired t test (n=3 biological replicates). * P<0.05.

### DETAILED DESCRIPTION

Aggressive PCa acquire mechanisms to escape immune detection and destruction ⁸ despite being infiltrated by immune cells and potentially immunogenic ^{9,10}. One of the key mechanisms is expression of programmed cell death I ligand (PD-L1) on tumor cells. Aggressive PCa expresses high levels of PD-L1, and its expression correlates with poor prognosis ^{11,12}. Although PD pathway blockade therapies have shown tremendous success in treating some tumors ¹³⁻¹⁶, only modest objective responses have been seen in aggressive PCa ¹⁷⁻²⁰. Importantly, however, elimination of PD-L1 expression in mouse NEPC model that is resistant to PD pathway blockade²¹ resulted in significant tumor suppression ²². Thus, it is urgent to understand the mechanisms that regulate PD-L1 expression in aggressive PCa to develop new therapeutic strategies.

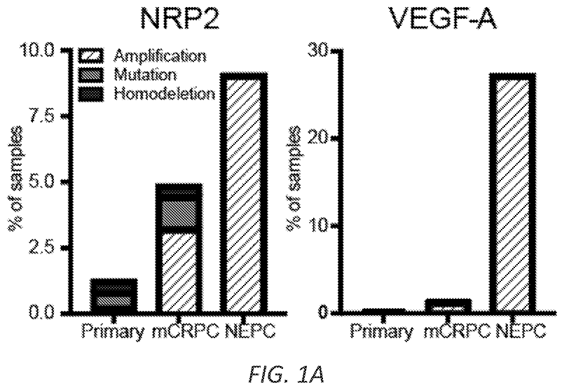

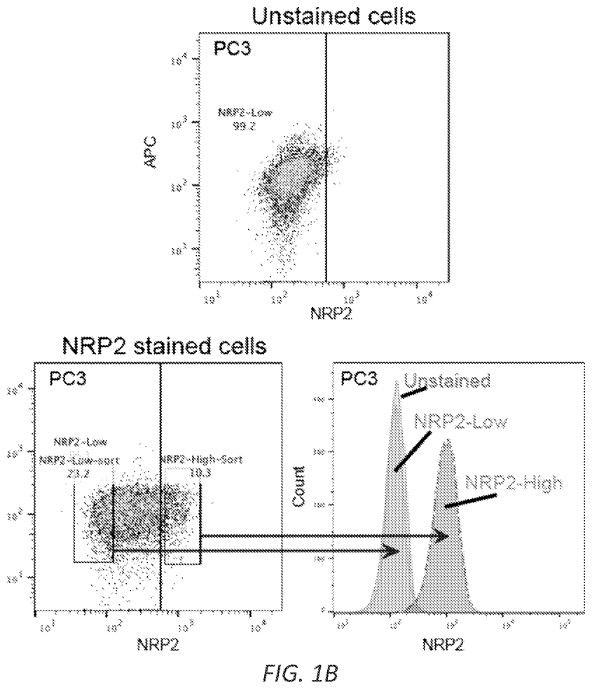

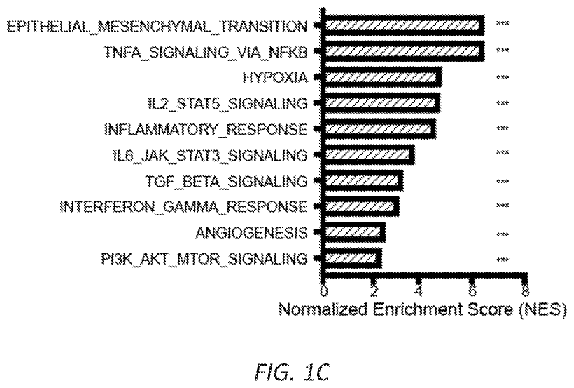

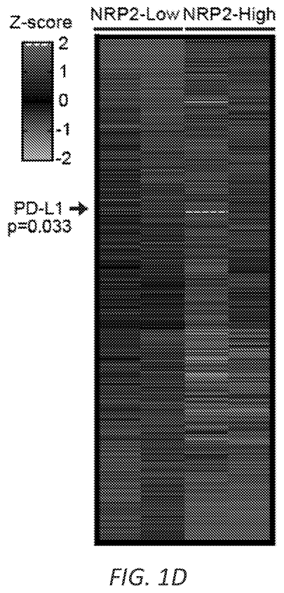

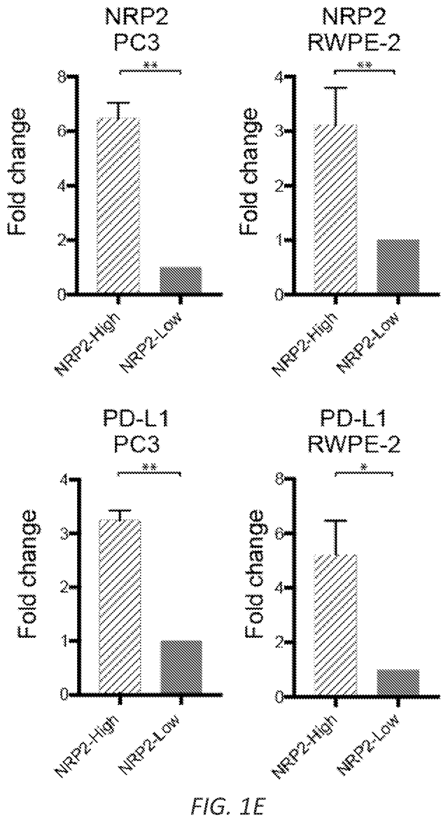

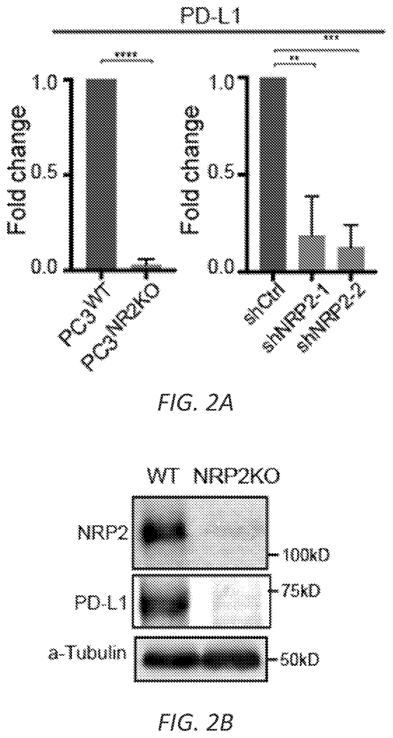

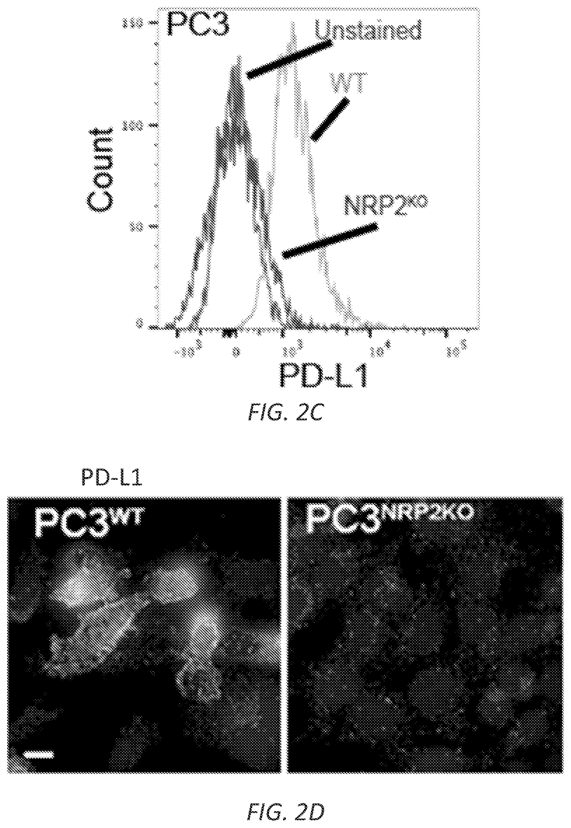

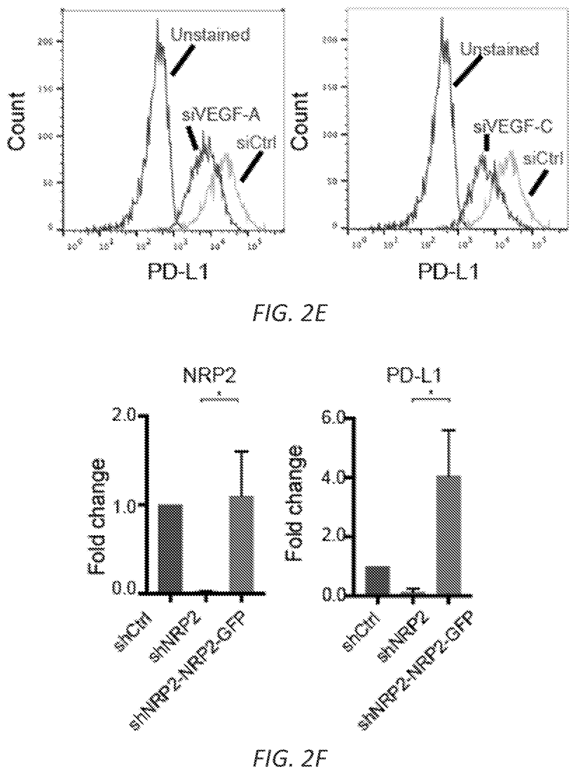

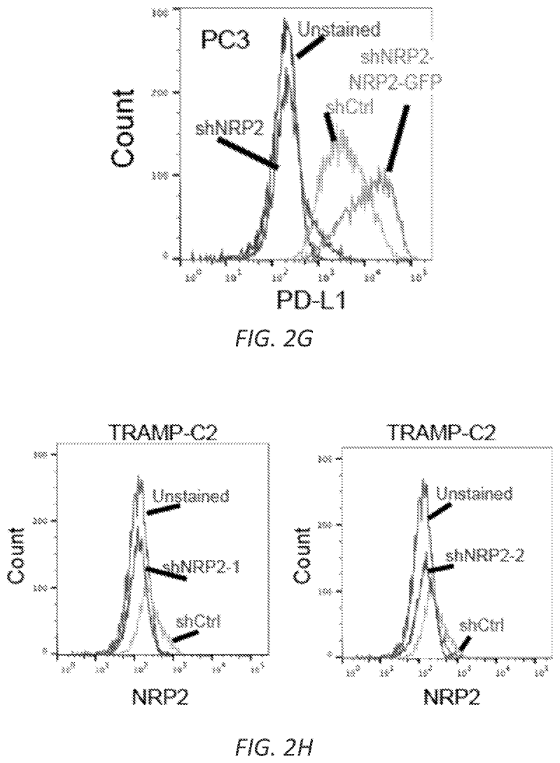

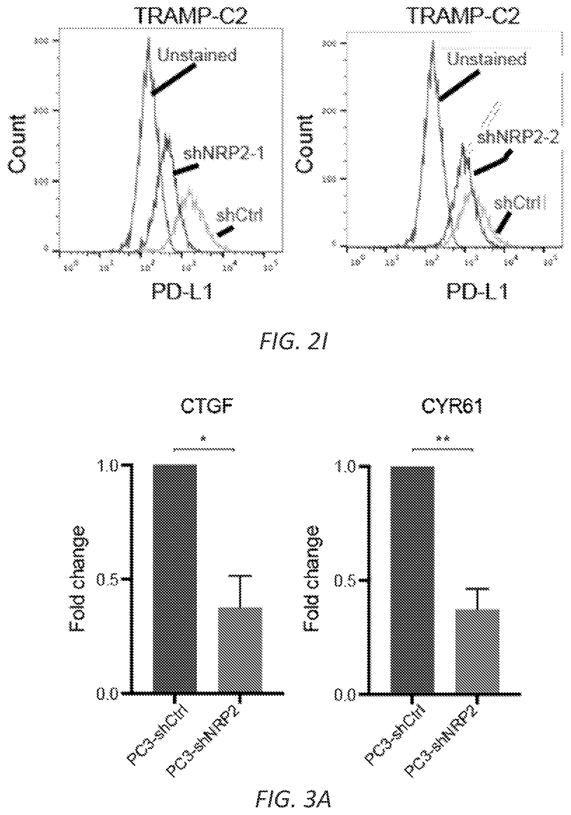

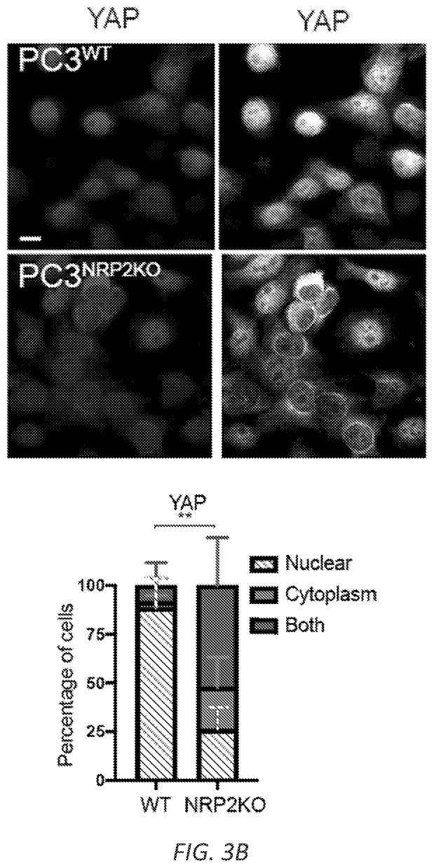

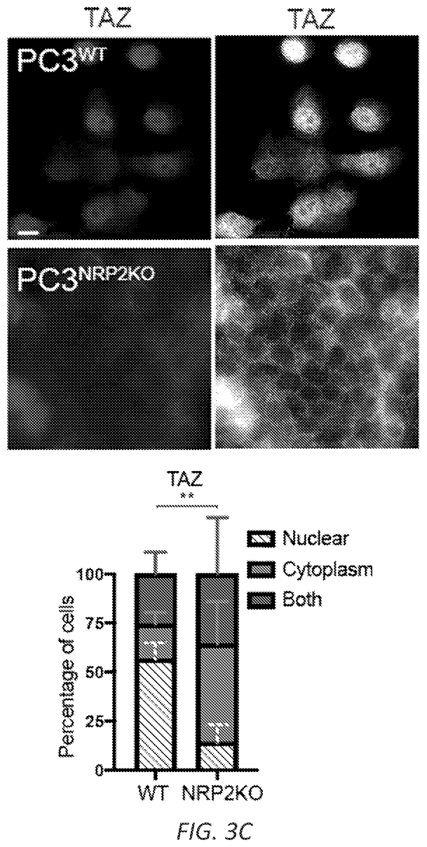

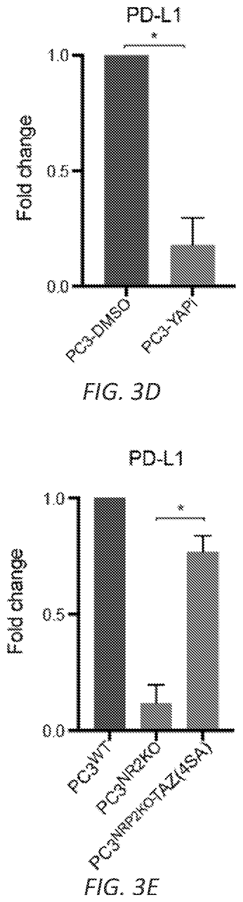

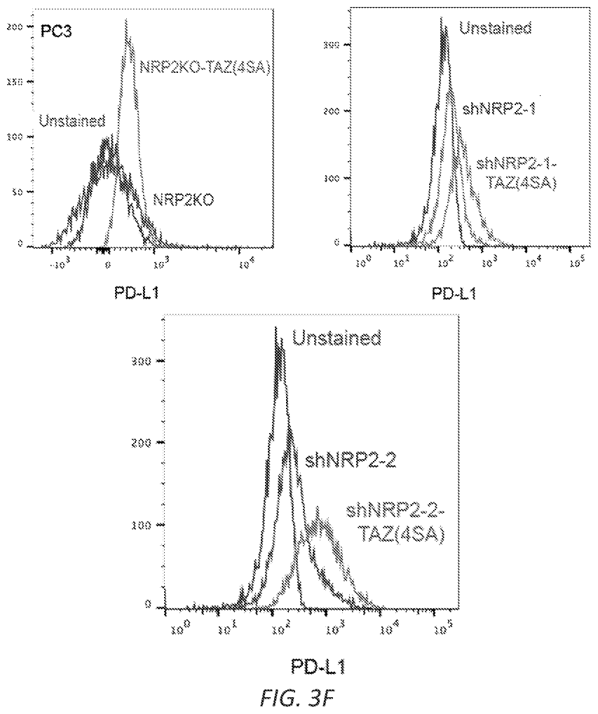

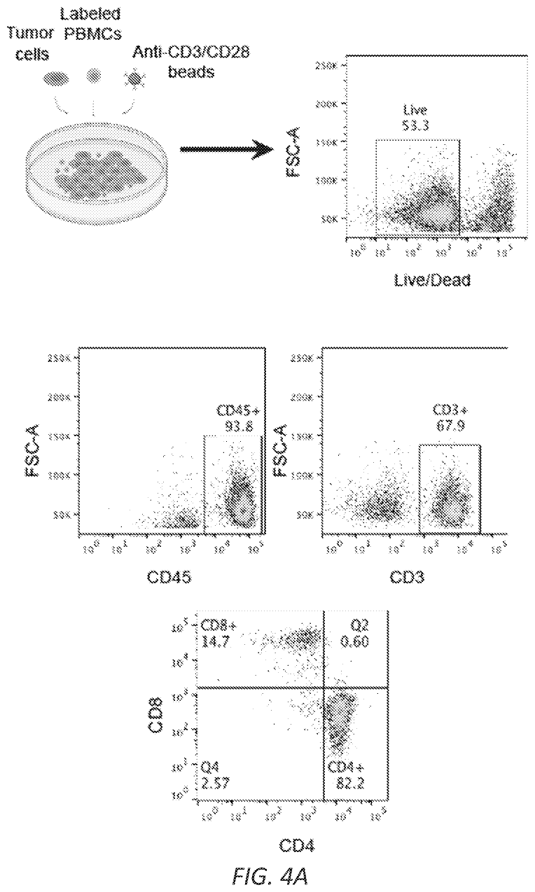

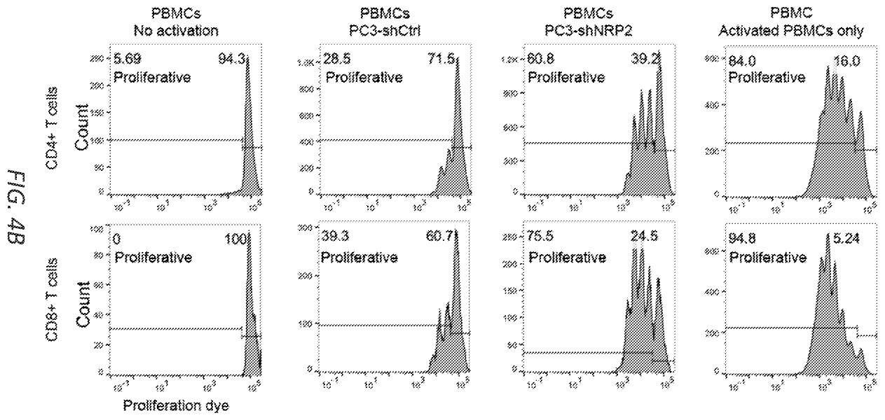

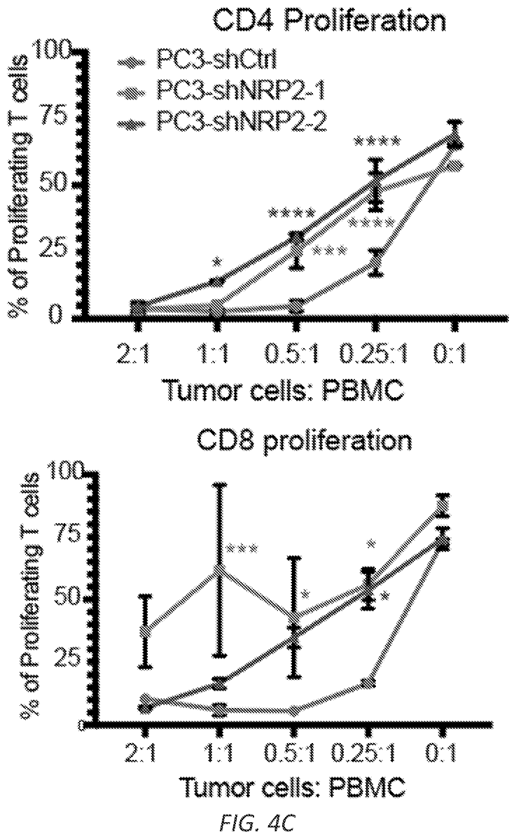

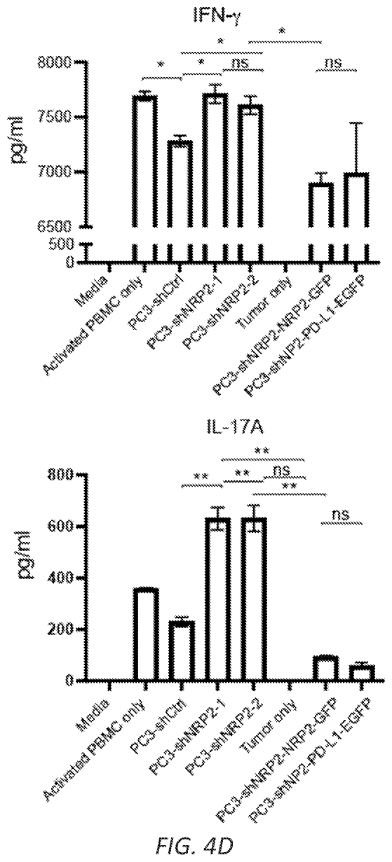

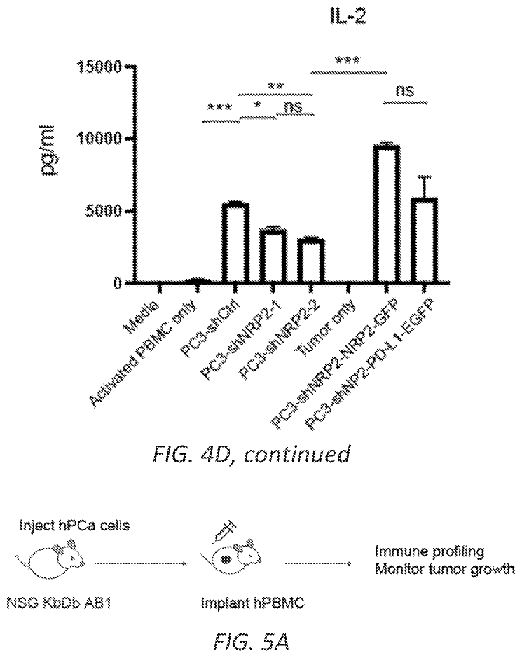

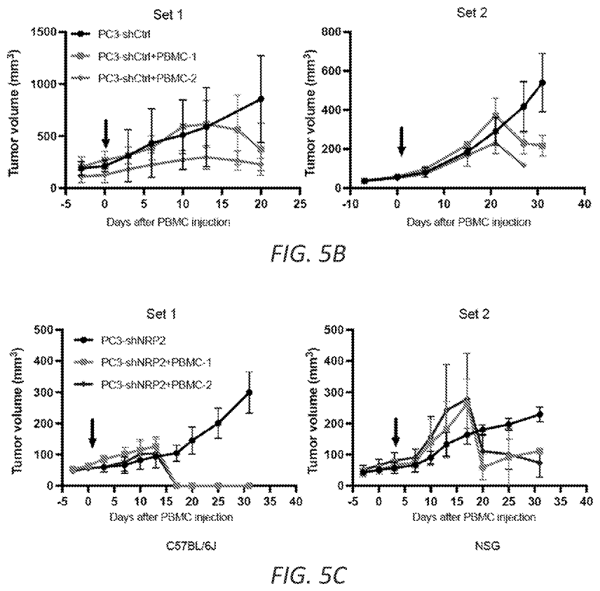

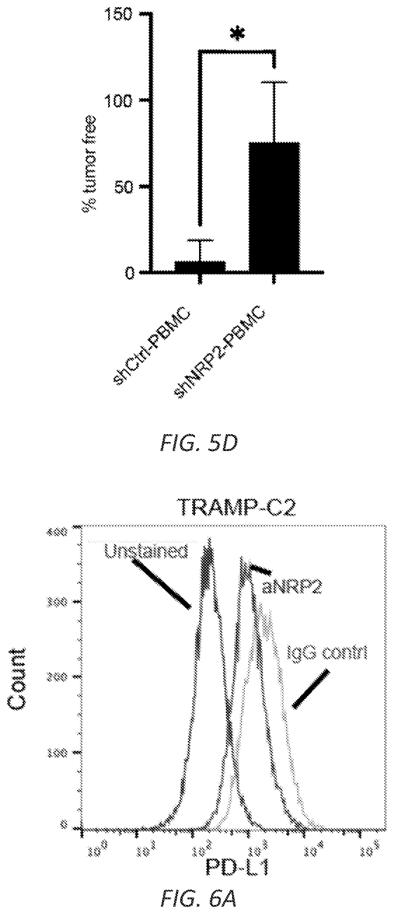

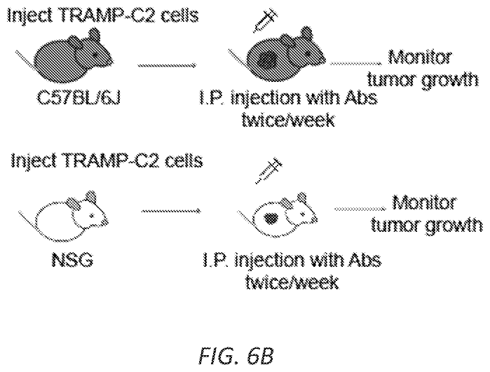

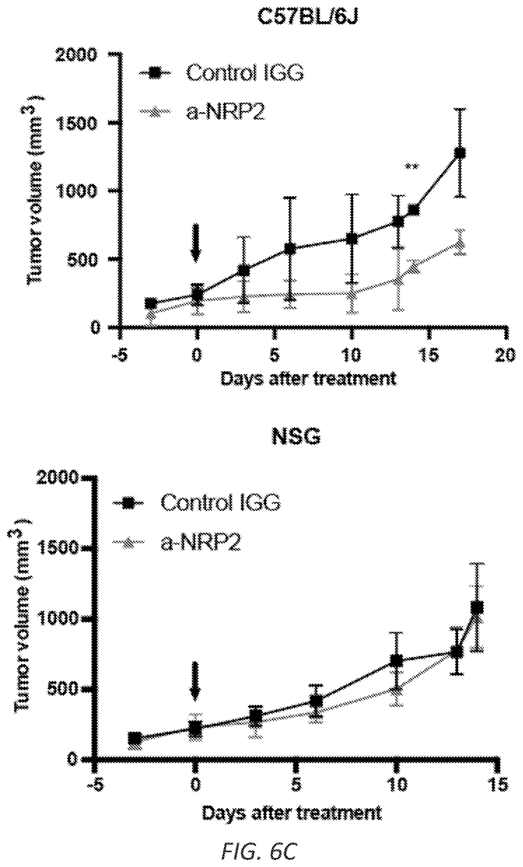

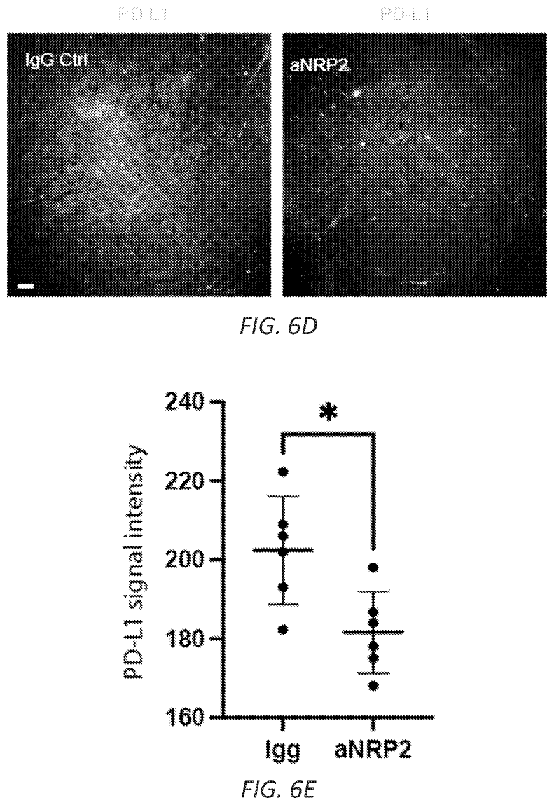

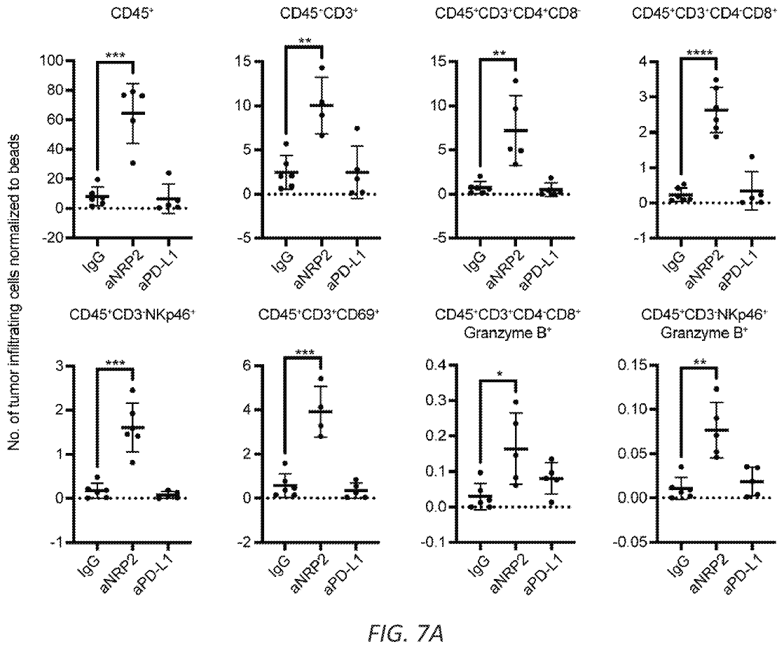

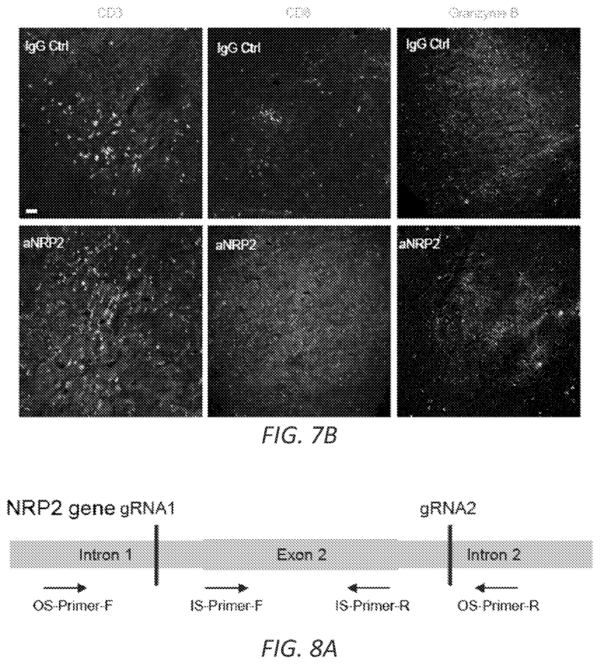

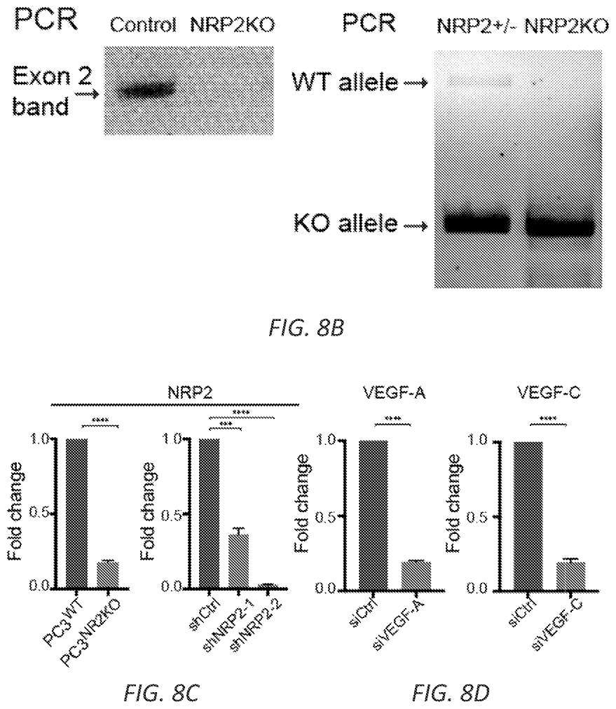

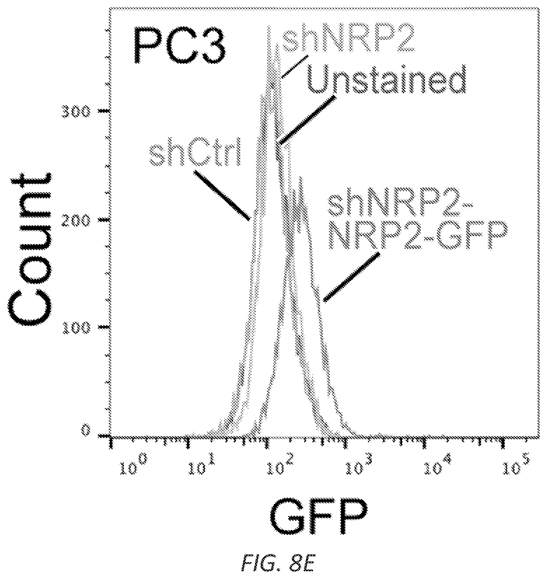

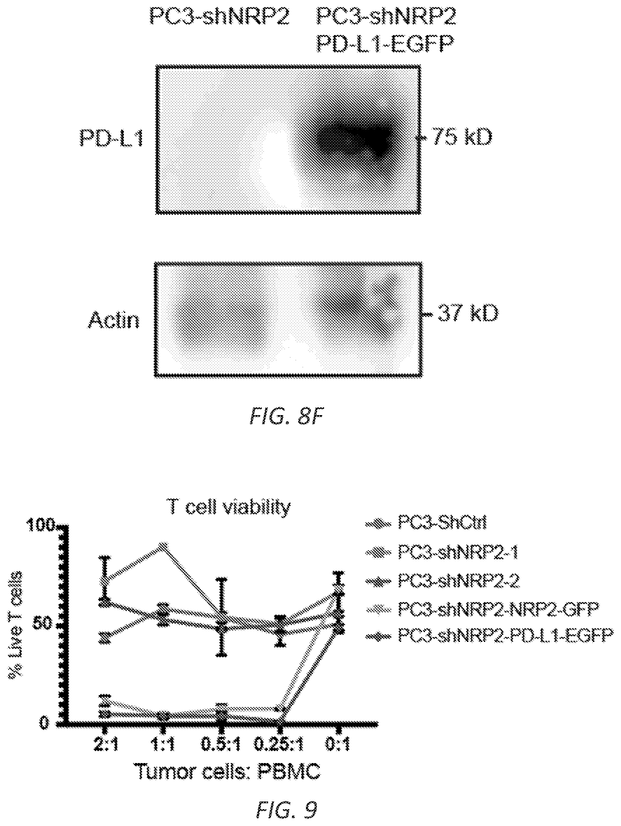

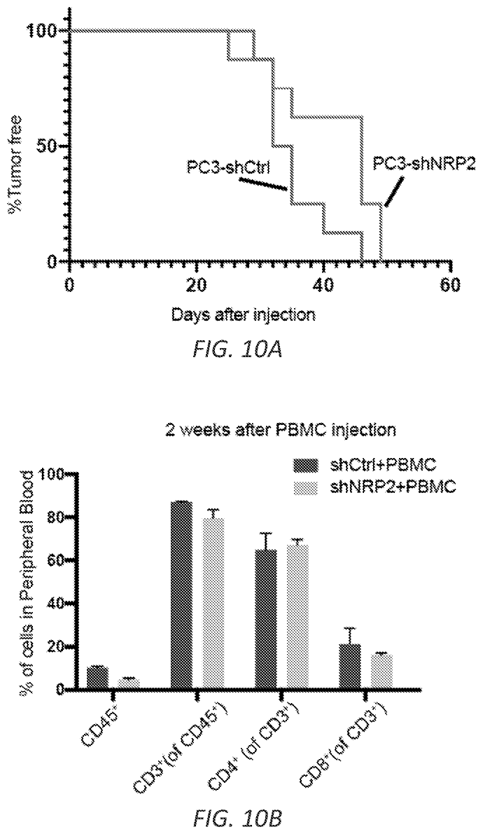

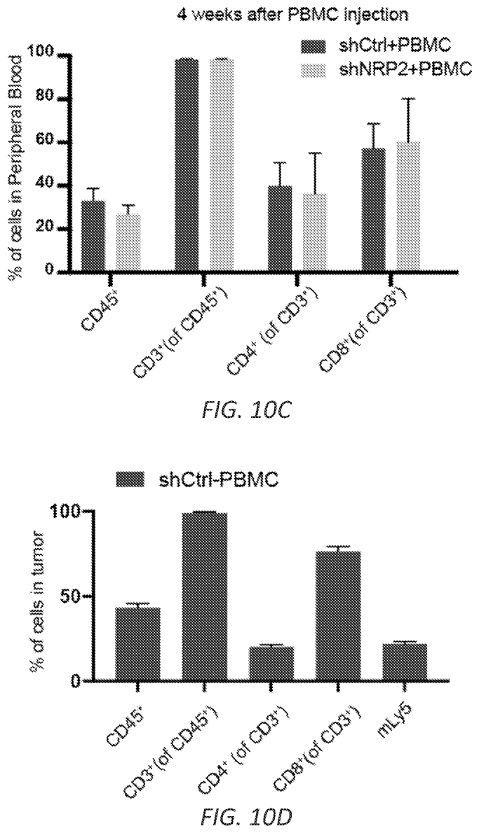

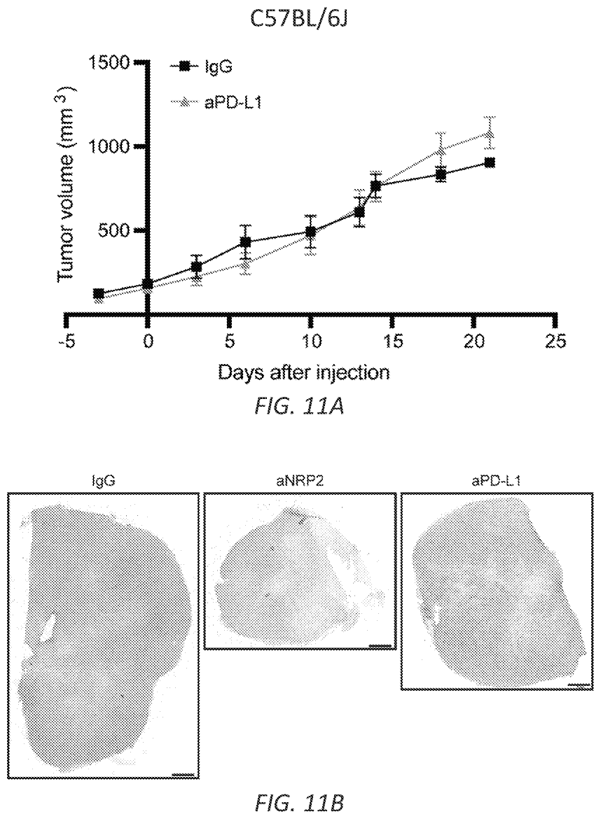

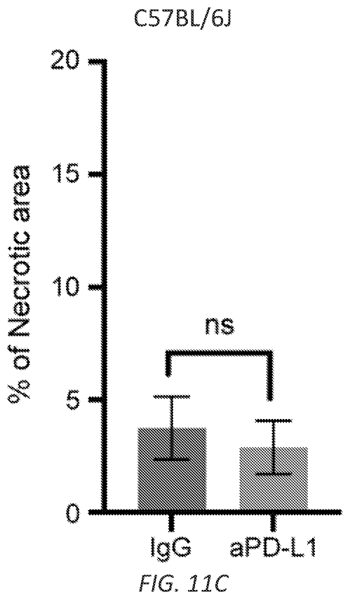

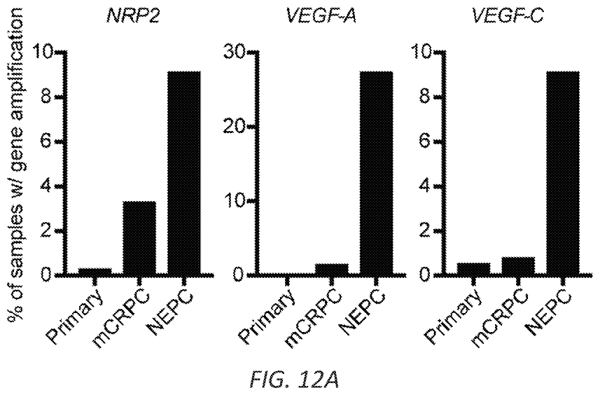

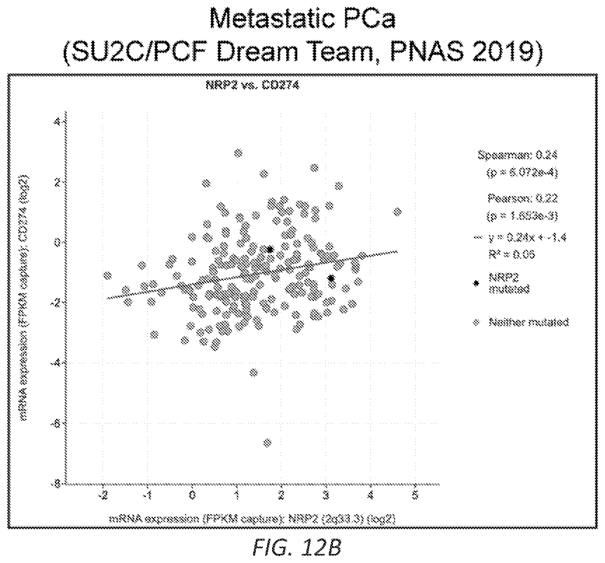

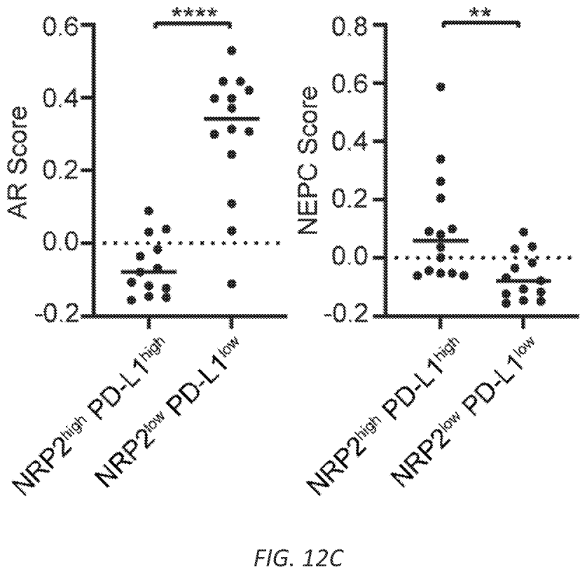

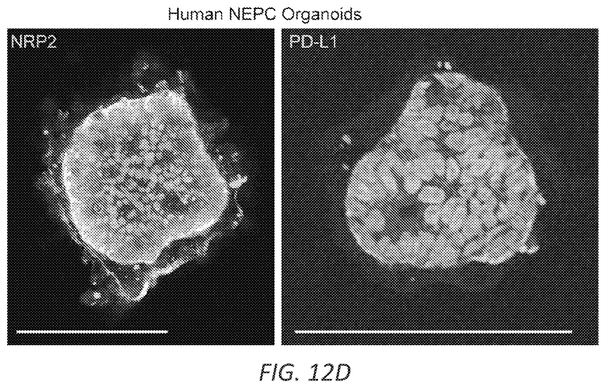

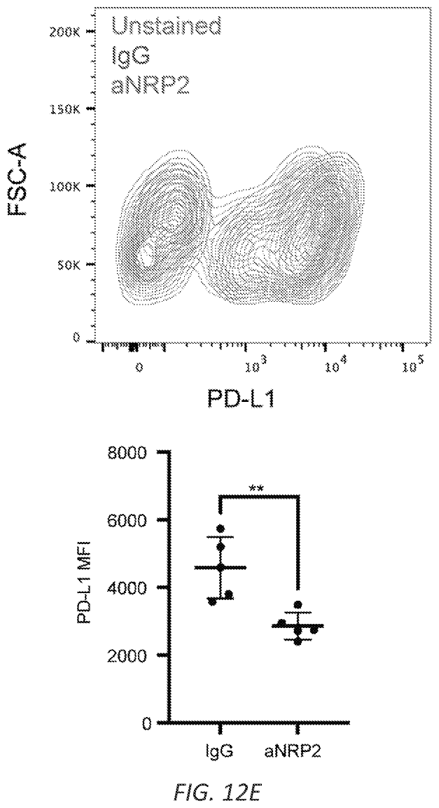

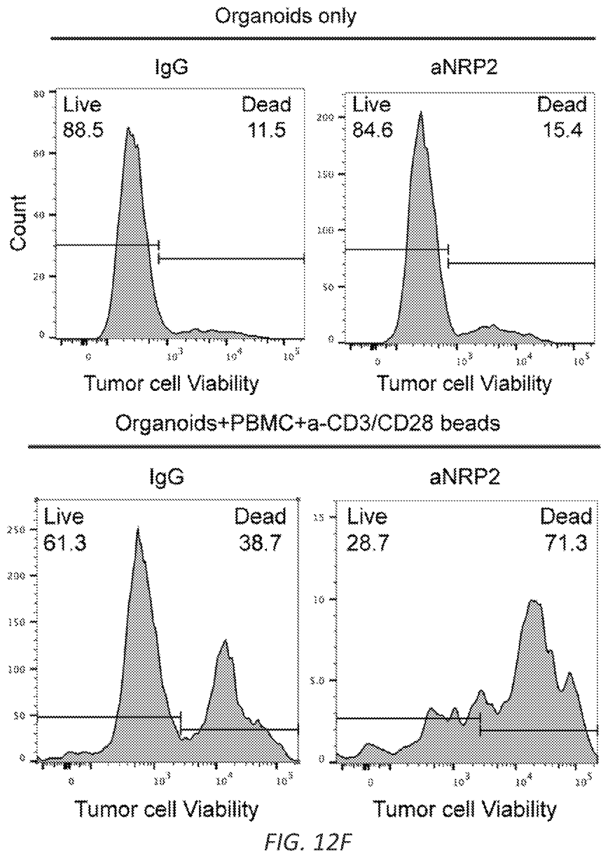

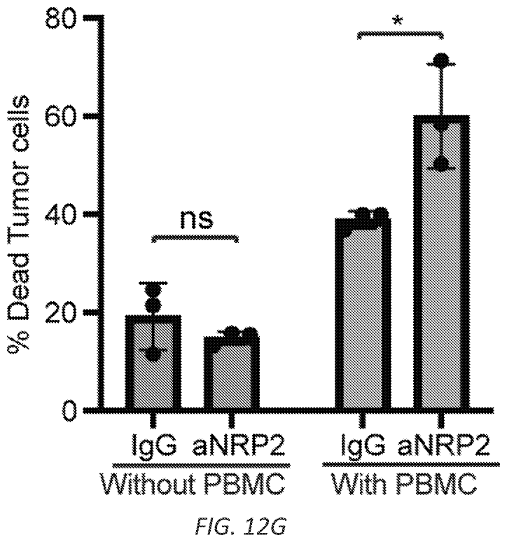

Neuropilin-2 (NRP2), a single pass transmembrane protein, is one of two NRPs (NRP1 and NRP2) that originally identified as receptors for axonal guidance factors termed the semaphorins during neurodevelopment ^{23,24} and can also function as VEGF receptors ²⁵. They are expressed on endothelial and tumor cells ²⁵, as well as immune cells ²⁶. The importance of NRP2 in cancer, especially carcinomas. has been revealed in many studies. A particularly striking observation is that the expression of NRP2 is low or absent in most adult epithelia that have been studied but is elevated in carcinomas that arise from these epithelia^{27,28}. More specifically, its expression is associated with more aggressive sub-types of specific cancers and it has been implicated in their aggressive behavior ²⁷⁻³². Studies on PCa exemplify these conclusions. NRP2 is not expressed in either normal prostate epithelium or prostatic intraepithelial neoplasia (PIN), but it is expressed in PCa ²⁷. Strikingly, we discovered that the genes for NRP2 and one of its ligands, VEGF-A, are significantly amplified in mCRPC and NEPC based on databases in cBioPortal (FIG. 1A). In fact. PTEN loss, which is associated with aggressive PCa ^{32,33}, induces NRP2 expression ²⁷, and it has been implicated in the aggressive behaviors of PCa cells such as drug resistance ^{29,34}. Further, NRP2 contribute to Hippo pathway transducer YAP/TAZ activation and promotes stem-like traits in cancer cells ³¹, which appear to be less responsive to PD pathway blockade than less stem-like cancer cells³⁴. While NRPs are expressed in various immune cells and regulate immune response ²⁶, the immune regulatory function of NRP2 in tumor cells is poorly understood. Importantly, NRP2 can be an effective therapeutic target in vivo using function-blocking Abs³⁵. These attributes make NRP2 an attractive therapeutic target in treating lethal PCa, a possibility that is being investigated actively.

### Methods of Treatment

Provided herein are compositions and methods for treating advanced prostate cancer, e.g., for treating metastatic castration resistant prostate cancer (mCRPC), e.g., neuroendocrine prostate cancer (NEPC) in a subject, e.g., a mammalian subject, preferably a human subject. Subjects are also referred to herein as “patients.” Neuroendocrine prostate cancer is an aggressive form of PCa that is associated with rapid progression, resistance to treatments and a very poor outcome with most patients surviving less than 1 year. One of the factors contributing to the aggressive behaviors of neuroendocrine prostate cancer is by changing the cancer cells to behave similar to stem cells. Importantly, treatments that can reverse stemness could sensitize NEPC cells to therapies [4, 6]. In some embodiments, the human subject has received at least 1 or 2 lines of systemic therapy for prostate cancer and has relapsed from the last systemic therapy, e.g., a systemic therapy such as hormonal therapy via surgical or chemical castration (LHRH agonist), chemotherapy, and/or radiopharmaceutical therapy.

As used in this context, to “treat” means to ameliorate at least one symptom of the cancer. For example, administration of a therapeutically effective amount of a compound described herein for treating mCRC or NEPC can result in a reduction in tumor size or decreased growth rate, a reduction in risk or frequency of reoccurrence, a delay in reoccurrence, a reduction in metastasis, increased survival, and/or decreased morbidity and mortality, inter alia.

Generally, the methods include administering a therapeutically effective amount of a treatment as described herein, to a subject who is in need of, or who has been determined to be in need of, such treatment. In some embodiments, the methods include administering a therapeutically effective amount of a treatment comprising a NRP2 inhibitor, optionally in combination with a treatment comprising an anti-angiogenic agent (e.g., a VEGF inhibitor, e.g., antibodies such as bevacizumab, ramucirumab, or ranibizumab; aflibercept: or Tyrosine kinase inhibitors (TKIs) such as sunitinib, pazopanib, sorafenib, nilotinib, axitinib, or dasatinib) (see, e.g., WO 2008/143665 or WO 2008/143666), and/or a treatment comprising chemotherapy, radiotherapy, and/or resection. In some embodiments, the methods further comprise administering a treatment comprising a chemotherapy, e.g., including one or more antimicrotubule agents including polymerizing agents (e.g., taxanes such as paclitaxel or docetaxel) and depolymerizing drugs (e.g., vinca alkaloids such as vincristine, vindesine, vinblastine, or vinorelbine), benzoylphenylureas (BPUs, e.g., NSC-639829), epothilones (e.g., ixabepilone) or estramustine phosphate); one or more platinum-based agents, e.g., cisplatin, oxaliplatin, or carboplatin; and/or one or more poly (ADP-ribose) polymerase inhibitors (PARPis, e.g., rucaparib, talazoparib, niraparib, or olaparib); and/or a dendritic cell vaccine (e.g., SIPULEUCEL-T) (Sutherland et al., Front Immunol. 2021 Mar. 29:12:641307). In some embodiments, the methods comprise administering a treatment as described herein in combination with at least one or two additional chemotherapeutic agents, e.g., etoposide, carboplatin, cisplatin, and/or docetaxel. E.g., at least two additional chemotherapeutic agents selected from etoposide+carboplatin: etoposide+cisplatin; and docetaxel+carboplatin.

Methods of identifying subjects with mCRPC or NEPC for treatment using the present methods are known in the art and can be performed by a skilled healthcare provider. See, e.g., Vlachostergios et al., Curr Oncol Rep. 2017 May; 19 (5): 32; Masone, Nature Reviews Urology 19 (2): 67 (2022); Conteduca et al., Eur J Cancer. 2019 November; 121:7-18; Beltran et al., Clin Cancer Res 20, 2846-2850 (2014): Rickman et al., Nat Med 23, 1-10, doi: 10.1038/nm.4341 (2017). In some embodiments, the methods include a step of identifying or selecting a subject as having mCRPC or NEPC, or identifying or selecting a subject who has been diagnosed as having mCRPC or NEPC. In some embodiments, the NEPC is characterized by the presence of cell morphology/histology and/or expression of one or more NEPC markers, e.g., one or more of synaptophysin, chromogranin A (CgA), neuron-specific enolase (NSE), and/or CD56. The methods can include determining the presence or absence of an NEPC in a tissue sample from the subject (e.g., by cell morphology/histology or immunohistochemistry); and administering a treatment to the subject as described herein, e.g., an antibody or antigen-binding fragment thereof, if the NEPC is present in the tissue sample.

In some embodiments, the methods include a step of identifying or selecting a subject, e.g., by determining a level of NRP2 expression in a sample comprising cells from the prostate tumor, and comparing the level of NRP2 in the sample to a reference level, and selecting a subject who has a cancer with a level of NRP2 expression above a reference level. In some embodiments, the methods include obtaining a tissue sample comprising cells from the prostate tumor from the subject, e.g., a liquid biopsy optionally a blood sample, a surgical sample, or other biopsy sample obtained from the subject, optionally a biopsy of prostate cancer tissue.

The presence and/or level of NEPC markers and/or NRP2 protein can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (particularly matrix-assisted laser desorption/ionization mass spectrometry (MALDI-MS) and surface-enhanced laser desorption/ionization mass spectrometry (SELDI-MS)) (Kim (2010) Am J Clin Pathol 134:157-162; Yasun (2012) Anal Chem 84 (14): 6008-6015; Brody (2010) Expert Rev Mol Diagn 10 (8):1013-1022; Philips (2014) PLOS One 9 (3):e90226; Pfaffe (2011) Clin Chem 57 (5): 675-687). The methods typically include revealing labels such as fluorescent, chemiluminescent, radioactive, and enzymatic or dye molecules that provide a signal either directly or indirectly. As used herein, the term “label” refers to the coupling (i.e, physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase. HRP) by reactivity with a detectable substance.

The presence and/or level of NEPC markers and/or NRP2 nucleic acids can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); gene arrays. RNAse protection assay; Northern blot; various types of nucleic acid sequencing (Sanger, pyrosequencing. NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48 (8): 1178-1185; Miranda (2010) Kidney International 78:191-199; Bianchi (2011) EMBO Mol Med 3:495-503; Taylor (2013) Front. Genet. 4:142; Yang (2014) PLOS One 9 (11):e110641); Nordstrom (2000) Biotechnol. Appl. Biochem. 31 (2):107-112; Ahmadian (2000) Anal Biochem 280:103-110. In some embodiments, high throughput methods, e.g., protein or gene chips as are known in the art (see, e.g., Ch.

12, Genomics, in Griffiths et al., Eds. Modern genetic Analysis, 1999, W. H. Freeman and Company: Ekins and Chu, Trends in Biotechnology. 1999, 17:217-218; MacBeath and Schreiber, Science 2000, 289 (5485):1760-1763; Simpson. Proteins and Proteomics: A Laboratory Manual, Cold Spring Harbor Laboratory Press: 2002; Hardiman, Microarrays Methods and Applications: Nuts & Bolts, DNA Press, 2003), can be used to detect the presence and/or level of NRP2.

In some embodiments, the presence and/or level of NEPC markers and/or NRP2 is comparable to or above the presence and/or level of NEPC markers and/or NRP2 in a disease reference, and the subject has one or more symptoms associated with mCRPC or NEPC, then the subject can be identified as having mCRPC or NEPC and optionally treated with a method described herein, or treated with a method comprising standard of care but not including immunotherapy.

Suitable reference values can be determined using methods known in the art, e.g., using standard clinical trial methodology and statistical analysis. The reference values can have any relevant form. In some cases, the reference comprises a predetermined value for a meaningful level of NEPC markers and/or NRP2, e.g., a control reference level that represents a normal level of NEPC markers and/or NRP2, e.g., a level in an unaffected subject or a subject who does not have and is not at risk of developing mCRPC or NEPC, and/or a disease reference that represents a level of the proteins associated with mCRPC or NEPC, e.g., a level in a subject having mCRPC or NEPC.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold. 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject has prostate cancer, but does not have or has not yet developed, or is not at risk of developing mCRPC or NEPC. In some cases it may be desirable that the control subject does not have prostate cancer.

A disease reference subject can be one who has (or who has an increased risk of developing) mCRPC or NEPC. An increased risk is defined as a risk above the risk of subjects in the general population.

Thus, in some cases the level of NEPC markers and/or NRP2 in a subject being greater than or equal to a reference level of NEPC markers and/or NRP2 is indicative of a clinical status (e.g., indicative of presence of mCRPC or NEPC). In other cases the level of NEPC markers and/or NRP2 in a subject being less than or equal to the reference level of NEPC markers and/or NRP2 is indicative of the absence of disease or normal risk of the disease. In some embodiments, the amount by which the level in the subject is the less than the reference level is sufficient to distinguish a subject from a control subject, and optionally is a statistically significantly less than the level in a control subject. In cases where the level of NEPC markers and/or NRP2 in a subject being equal to the reference level of NEPC markers and/or NRP2, the “being equal” refers to being approximately equal (e.g., not statistically different).

The predetermined value can depend upon the particular population of subjects (e.g., human subjects) selected. For example, an apparently healthy population will have a different ‘normal’ range of levels of NRP2 than will a population of subjects which have prostate cancer, are likely to have, or are at greater risk to have, a disorder described herein. Accordingly, the predetermined values selected may take into account the category (e.g., sex, age. health, risk, presence of other diseases) in which a subject (e.g., human subject) falls. Appropriate ranges and categories can be selected with no more than routine experimentation by those of ordinary skill in the art.

### NRP2 Inhibitors

The present methods and compositions can include the use of NRP2 inhibitors, i.e., agents that bind directly to NRP2 and inhibit its activity (e.g., inhibits NRP2-VEGF signaling or binding of VEGF to NRP2), or bind to NRP2 encoding nucleic acids (e.g., mRNA or genomic DNA) and reduce or eliminate expression of NRP2. See, e.g., Wang et al., Cancer Lett. 2018 Apr. 1; 418:176-184; Zhao et al., J Drug Target. 2021 February; 29 (2): 155-167.

Such agents can include antibodies (or antigen-binding fragments thereof), inhibitory peptides, or small molecules that bind to NRP2 proteins, or and inhibitory nucleic acids that bind to NRP2 nucleic acids. Exemplary sequences for human NRP2 are provided in Table A.

TABLE A

| Nucleic acid | Protein | Name |
|---|---|---|
| NM_201266.2 | NP_957718.1 | neuropilin-2 isoform 1 precursor |
| NM_003872.3 | NP_003863.2 | neuropilin-2 isoform 2 precursor |
| NM_201279.2 | NP_958436.1 | neuropilin-2 isoform 3 precursor |
| NM_018534.4 | NP_061004.3 | neuropilin-2 isoform 4 precursor |
| NM_201267.2 | NP_957719.1 | neuropilin-2 isoform 5 precursor |
| NM_201264.2 | NP_957716.1 | neuropilin-2 isoform 6 precursor |

Antibodies. Inhibitory Peptides, and Small Molecules

The term “antibody” as used herein refers to an immunoglobulin molecule or an antigen-binding portion thereof. Examples of antigen-binding portions of immunoglobulin molecules include F(ab) and F(ab′): fragments, which retain the ability to bind antigen. The antibody can be polyclonal, monoclonal, recombinant, chimeric, de-immunized or humanized, fully human, non-human, (e.g., murine), or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region. Antibodies (or antigen-binding fragments thereof) that bind to NRP2 can be generated using methods known in the art; see, e.g., Harlow et, al., editors, Antibodies: A Laboratory Manual (1988); Goding, Monoclonal Antibodies: Principles and Practice, (N.Y. Academic Press 1983); Howard and Kaser, Making and Using Antibodies: A Practical Handbook (CRC Press; 1st edition, Dec. 13, 2006); Kontermann and Dübel, Antibody Engineering Volume 1 (Springer Protocols) (Springer; 2nd ed., May 21, 2010); Lo, Antibody Engineering: Methods and Protocols (Methods in Molecular Biology) (Humana Press; Nov. 10, 2010); and Dübel, Handbook of Therapeutic Antibodies: Technologies, Emerging Developments and Approved Therapeutics, (Wiley-VCH: 1 edition Sep. 7, 2010). See also, e.g., Hanak et al., Adv Exp Med Biol. 2016:917:11-22: Tiller and Tessier, Annu Rev Biomed Eng. 2015:17:191-216; Waldmann, Methods Mol Biol. 2019:1904: 1-10; and Yang et al., Monoclon Antib Immunodiagn Immunother. 2015 October: 34 (5): 354-9. Alternatively, the antibodies can be obtained commercially, e.g., from Abcam, ABclonal Technology, Abnova Corporation, Affinity Biosciences, Alomone Labs, Ltd., antibodies-online. Beijing Solarbio Science & Technology, Bioassay Technology Laboratory, Biorbyt, Bioss Inc., BosterBio, Cell Signaling Technology, Creative Biolabs, Creative Diagnostics, ECM Biosciences, G Biosciences, Gene Tex, LifeSpan BioSciences, MilliporeSigma, MyBioSource.com, Novus Biologicals, NSJ Bioreagents, OriGene Technologies, ProSci, Inc, Proteintech Group Inc, R&D Systems (see, e.g., Yasuoka et al., J Clin Endocrinol Metab. 2011 November; 96 (11):E1857-61), RayBiotech, Santa Cruz Biotechnology, Inc., Sino Biological, Inc., Thermo Fisher Scientific, or United States Biological. In some embodiments, the antibody is an antibody or antigen-binding fragment thereof that specifically binds to a human neuropilin-2 (NRP2) polypeptide (anti-NRP2 antibody) as described in Yang et al., Monoclon Antib Immunodiagn Immunother. 2015 October; 34 (5):354-9 (NRP2 MAb); Wang et al., Front Oncol. 2021 Jul. 15;11:657008 (N2E4); in US20210163606, e.g., aNRP2-10v10, aNRP2-11v7, or aNRP2-28, preferably aNRP2-10, e.g., aNRP2-10v10, or aNRP2-28, described therein: in US20190309076, e.g., 8v2 (aNRP2-8) #1328, 9v2 (aNRP2-9) #1329, 10v2 (aNRP2-10) #1330, 14v2. 11v2 (aNRP2-11) #1331, 14v2 (aNRP2-14), #1344, 15v2 (aNRP2-15) #1347, described therein; or in WO 2008/143665 (e.g., YW68.4.2, YW68.4.2.36, or YW 126.20) or WO 2008/143666 (YW107.4.87)). See also Appleton et al., EMBO J. 2007 Nov. 28: 26 (23): 4902-12, WO 2008/143665, and WO 2008/143666, which provide crystal structures and identifies portions of the NRP2 protein that are important for antibody inhibition. In some embodiments, the antibodies or antigen-binding fragments thereof specifically binds to a human neuropilin-2 (NRP2) polypeptide at an epitope in the neuropilin b1 domain of NRP2 Preferably, the antibodies or antigen-binding fragments thereof specifically inhibit NRP2-VEGF signaling. In some embodiments, the antibody is a humanized monoclonal antibody that comprises a human IgG4 Fc domain. See, e.g., US 20210163606. Optionally the IgG4 domain includes Ser228Pro and Leu235Ala mutations to attenuate the effector functions of the Fc region (see U.S. Pat. No. 7,030,226).

In some embodiments, the antibody or antigen-binding fragment thereof comprises a heavy chain variable region (V_{H}) sequence that comprises complementary determining region (CDR) V_{H}CDR1, V_{H}CDR2, and V_{H}CDR3 sequences set forth in SEQ ID NOs: 1-3, respectively, and a light chain variable region (V_{L}) sequence that comprises complementary determining region V_{L}CDR1, V_{L}CDR2, and V_{L}CDR3 sequences set forth in SEQ ID NOs: 4-6, respectively: or a V_{H} sequence that comprises V_{H}CDR1, V_{H}CDR2, and V_{H}CDR3 sequences set forth in SEQ ID NOs: 7-9, respectively, and V_{L} sequence that comprises V_{L}CDR1, V_{L}CDR2, and V_{L}CDR3 sequences set forth in SEQ ID NOs: 10-12, respectively. In some embodiments, the V_{H} sequence comprises a sequence at least 80, 85, 90, 95, 97, 98, 99, or 100% identical to SEQ ID NO: 13, and the V_{L} sequence comprises a sequence at least 80, 85, 90, 95, 97, 98, 99, or 100% identical to SEQ ID NO: 14; or the V_{H} sequence comprises a sequence at least 80, 85, 90, 95, 97, 98, 99, or 100% identical to SEQ ID NO: 15, and the VL, sequence comprises a sequence at least 80, 85, 90, 95, 97, 98, 99, or 100% identical to SEQ ID NO: 16; or the V_{H} sequence comprises a sequence at least 80, 85, 90, 95, 97, 98, 99, or 100% identical to SEQ ID NO: 17, and the V_{L} sequence comprises a sequence at least 80, 85, 90, 95, 97, 98, 99, or 100% identical to SEQ ID NO: 18. See US20210163606; the CDRs correspond to SEQ ID NOs: 19-24 (2-10v10) and 63-68 (2-28v2/4), and the V_{H}/V_{L} I correspond to SEQ ID NOs: 49/50 (2-10v10), 71/72 (28v2), and 73/74 (28v4).

TABLE A1
Exemplary CDR Sequences

| | | SEQ ID |
|---|---|---|
| | | NO: |
| aNRP2-10v10 | | |
| VH CDR1 | GFNIKDYYVH | 1 |
| VL CDR2 | RIDVEDDETK | 2 |
| | YAPKFQG | |
| VH CDR3 | PIYGAREAWFAY | 3 |
| VL CDRI | TASSSVSSSYLH | 4 |
| VL CDR2 | RTSNLAS | 5 |
| VL CDR3 | HQYYRSPPT | 6 |
| aNRP2-28v2/4 | | |
| VH CDR1 | GFSLSTYSIS | 7 |
| VH CDR2 | IIGDAGGIIY | 8 |
| | ATWAKS | |
| VH CDR3 | DGTAFDI | 9 |
| VL CDRI | QASQSIYSKLG | 10 |
| VL CDR2 | RASTLAS | 11 |
| VL CDR3 | QQDYSYINVDNI | 12 |

TABLE A2
Exemplary Polypeptide Sequences

| | | SEQ ID NO: |
|---|---|---|
| aNRP2-10v10 | | |
| Heavy chain | EVQLVQSGAEVKKPGATVKISCKVSGFNIKDY | 13 |
| variable | YVHWVQQAPGKGLEWMGRIDVEDDETKYAPK | |
| region (VH) | FQGRVTITADTSTDTAYMELSSLRSEDTAVY | |
| | YCATPIYGAREAWFAYWGQGTLVTVSS | |
| Light chain | DIQMTQSPSSLSASVGDRVTITCTASSSVSSSY | 14 |
| variable | LHWYQQKPGKAPKLLIYRTSNLASGVPSRFSGS | |
| region (VL) | GSGTDFTLTISSLQPEDFATYYCHQYYRSPPTF | |
| | GGGTKVEIK | |
| aNRP2-28v2 | | |
| Heavy chain | QSVKESEGGLFKPTDTLTLTCTVSGFSLSTYSI | 15 |
| variable | SWVRQAPGNGLEWIGIIGDAGGIIYATWAKSRS | |
| region (VH) | TITRSTALNTVTLKMTGLTAADTATYFCARDGT | |
| | AFDIWGPGTLVTVSS | |
| Light chain | AYDMTQTPASVEVVVGGTVTIKCQASQSIYSK | 16 |
| variable | LGWYQQKPGQPPKLLIYRASTLASGVSSRFKG | |
| region (VL) | SGSGTEYTLTISGVQCDDAATYYCQQDYSYIN | |
| | VDNIFGGGTEVVVK | |
| aNRP2-28v4 | | |
| Heavy chain | QSVKESEGGLFKPTDTLTLTCTVSGFSLSTYSIS | 17 |
| variable | WVRQAPGNGLEWIGIIGDAGGIIYATWAKSRSTI | |
| region (VH) | TRSTALNTVTLKMTGLTAADTATYFCARDGTAFD | |
| | IWGPGTLVTVSS | |
| Light chain | AYDMTQTPASVEVVVGGTVTIKCQASQSIYSKL | 18 |
| variable | GWYQQKPGQPPKLLIYRASTLASGVSSRFKGSG | |
| region (VL) | SGTEYTLTISGVQADDAATYYCQQDYSYINVDN | |
| | IFGGGTEVVVK | |

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

Peptide inhibitors of NRP2 are also known in the art, e.g., peptides obtained by mutagenesis of the soluble NRP2 B domain, e.g., B-NRP2 R287E N290D (MutB-NRP2) as described in Geretti et al., Mol Cancer Res. 2010 August: 8 (8): 1063-73 and Geretti et al., J Biol Chem. 2007 Aug. 31;282 (35): 25698-707; US20020132774; and US20100267140.

Small molecule inhibitors of NRP2 include Zafirlukast. Actinomycin D, and Dihydrexidine, which inhibit AP-VEGF-C/Nrp2-b1b2 binding (Parker and Kooi, Anal Biochem. 2014 May 15; 453:4-6); benzamidine-based inhibitors of VEGF-C binding to Neuropilin-2 (Said et al., Bioorganic Chemistry Volume 100, July 2020, 103856 https://doi.org/10.1016/j.bioorg.2020.103856.

### Inhibitory Nucleic Acids

Inhibitory nucleic acids useful in the present methods and compositions include antisense oligonucleotides (ASOs), single- or double-stranded RNA interference (RNAi) compounds such as siRNA or shRNA, modified bases/locked nucleic acids (LNAs), peptide nucleic acids (PNAs), and other oligomeric compounds or oligonucleotide mimetics that hybridize to at least a portion of the target NRP2 nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA): a micro, interfering RNA (miRNA): a small, temporal RNA (stRNA); or a short, hairpin RNA (shRNA); small RNA-induced gene activation (RNAa): small activating RNAs (saRNAs), or combinations thereof. See, e.g., WO 2010040112.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol . . . 1990, 215, 403-410; Zhang and Madden, Genome Res., 1997, 7, 649-656), e.g., using the default parameters.

Once one or more target regions, segments or sites have been identified, e.g., within a target sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this disclosure, hybridization means hydrogen bonding. which may be Watson-Crick, Hoogsteen or reversed Hoogsteen hydrogen bonding. between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C., in 750 mM NaCl, 75 mM trisodium citrate, and 1% SDS. In a more preferred embodiment, hybridization will occur at 37° C., in 500 mM NaCl, 50 mM trisodium citrate, 1% SDS. 35% formamide, and 100 μg/ml denatured salmon sperm DNA (ssDNA). In a most preferred embodiment, hybridization will occur at 42° C., in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C., in 30 mM NaCl, 3 mM trisodium citrate, and 0.1% SDS. In a more preferred embodiment, wash steps will occur at 42° C., in 15 mM NaCl, 1.5 mM trisodium citrate, and 0.1% SDS. In a more preferred embodiment, wash steps will occur at 68° C., in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180. 1977): Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987. Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215. 403-410: Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

### Modified Inhibitory Nucleic Acids

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA: DNA or RNA: RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., . Stanton et al., Nucleic Acid Ther. 2012. 22:344-359: Nowotny et al., Cell, 121:1005-1016, 2005; Kurreck, European Journal of Biochemistry 270: 1628-1644. 2003; FLuiter et al., Mol Biosyst. 5 (8): 838-43, 2009). In some embodiments, the oligonucleotide is a mixmer (includes alternating short stretches of LNA and DNA: Naguibneva et al., Biomed Pharmacother. 2006 November; 60 (9): 633-8: Ørom et al., Gene. 2006 May 10; 372 ( ) 137-41). Representative United States patents that teach the preparation of such hybrid structures comprise, but are not limited to. U.S. Pat. Nos. 5,013,830:5,149,797:5, 220.007:5.256.775: 5,366,878:5,403,711:5,491, 133:5,565,350; 5,623,065; 5,652,355; 5,652,356; and 5,700,922, each of which is herein incorporated by reference.

### Dosage

A therapeutically effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week: including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Pharmaceutical Compositions

The methods described herein include pharmaceutical compositions comprising or consisting of NRP2 inhibitors as described herein as an active ingredient, and the use thereof in treating mCRPC or NEPC in a subject in need thereof, or in the preparation of a medicament for treating mCRPC or NEPC in a subject in need thereof.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., anti-angiogenic agents.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose, pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water. Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature; a binder such as microcrystalline cellulose, gum tragacanth or gelatin; an excipient such as starch or lactose, a disintegrating agent such as alginic acid. Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol . . . 88 (2). 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration. Also provided are kits, e.g., patient care kits, that include means for determining the presence or absence of a neuroendocrine prostate cancer (NEPC) in a tissue sample from a human patient with prostate cancer; and a composition as described herein, e.g., comprising an NRP2 inhibitor, e.g., an antibody or an antigen-binding fragment thereof that specifically binds to a human neuropilin-2 (NRP2) polypeptide, preferably at an epitope in the neuropilin b1 domain of NRP2. In some embodiments, means for determining the presence or absence of a neuroendocrine prostate cancer (NEPC) in a tissue sample from a human patient can include reagents for performing an assay as described herein, e.g., immunohistochemistry (IHC) on one or more NEPC markers (e.g., synaptophysin, chromogranin A (CgA), neuron-specific enolase (NSE), and/or CD56) and/or NRP2. The tissue sample can be, e.g., a liquid biopsy optionally a blood sample, a surgical sample, or other biopsy sample obtained from the patient, optionally a biopsy of prostate cancer tissue.

### Examples

The invention is further described in the following examples, which do not limit the scope of the invention described in the claims.

### Materials and Methods

The following materials and methods were used in the Examples below.

Reagents, antibodies and cell culture. PC3 cells were gifts from Dr. Anne Cress from the University of Arizona Cancer center. RWPE-2, TRAMP-C2 cells were obtained from the American Type Culture Collection. All cell lines were maintained at 37° C., with 5% CO₂ atmosphere. Compounds were added to and maintained in complete growth medium unless otherwise specified. All cells were checked quarterly for mycoplasma.

PBMCs were isolated from human blood from healthy volunteers after informed consent was obtained and all procedures related to human samples were performed according to the guidelines of Institute Biosafety Committee (IBC) of University of Massachusetts Medical School. Blood was collected into 150 ml Heparin-anticoagulated tubes and the mononuclear cells were collected by Ficoll density gradient centrifugation overlay method with 5 hours of collection of blood. Whole blood was mixed with an equal volume of RMPI-1640 (Gibco, Thermo fisher Scientific, USA) and the peripheral blood mononuclear cells were isolated by centrifugation at 1200 rpm for 30 mins at room temperature through Ficoll-Hypaque (Catalog #H889-500 ml. Sigma). The isolated buffy coat containing the mononuclear cells was washed twice with RPMI-1640 and the cell count was determined by trypan blue exclusion method using a hemocytometer.

The following antibodies and reagents were used: anti-a-tubulin monoclonal DMIA (Thermo Fisher Scientific), APC anti-human CD274 (B7-H1, PD-L1) antibody (Biolegend, 329708), anti-human CD274 (PD-L1, B7-H1) monoclonal Antibody (M1H1) (eBioscience™ Invitrogen, 14-5983-82), anti-mouse-PD-L1 (abcam, ab213480). PE Anti-PD-L1 antibody [10F.9G2] (Abcam, ab210189), anti-NRP2 antibody (Santa Cruz, sc-13117), anti-NRP2 antibody (R&D Systems, AF2215), anti-NRP2 antibody (aTyr, aNRP2-28). Alexa Fluor® 700 Mouse Anti-Human CD45 (BD Bioscience. 560566), APC Mouse Anti-Human CD4 (BD Bioscience, 555349), anti-TAZ antibody (BD Biosciences, 560235), anti-YAP antibody (Santa Cruz, sc-10199). PerCP/Cyanine5.5 anti-human CD3 Antibody (Biolegend, 300430), Brilliant Violet 605TM anti-human CD8a Antibody (Biolegend, 301040) Secondary antibodies were purchased from Invitrogen.

The following reagents were used: CellTrace™ Violet Cell Proliferation Kit (Thermo Fisher, C34571), eBioscience™ Fixable Viability Dye eFluor™ 780 (Invitrogen, 65-0865-14). Verteporfin was purchased from Tocris and used at a concentration of 2 μM for 24 hours.

Generation of engineered cell lines. To generate NRP2 knockout cells, we used Alt-R™ CRISPR-Cas9 System (IDT). The following gRNAs were used: (Human NRP2-g1: GCTCTTACTCAACTAGCAGA (SEQ ID NO:19), Human NRP2-g2: GTACCCTGCAACCCAAAGCA (SEQ ID NO:20)). The following reagents were purchased from IDT: Alt-RIM CRISPR erRNA, 2 nmol), CRISPR-Cas9 tracrRNA (IDT, Cat. 1072532), and Cas9 Nuclease (IDT, Alt-RTM S.p. Cas9 Nuclease 3NLS, Cat. 1074181) and were used to assemble Cas9: crR.NA: tracrRNA ribonucleoprotein (RNP) complex. The RNP complexes were transfected in cells using Nucleofector Device (Amaxa). Single cells were sorted into 96-well plates with 1 cell/well. Cells were screened for homozygous NRPR knockout using PCR from gemonic DNA. The following primers were used:

(NRP2-screen1-F:
(SEQ ID NO: 21)
ACTACCCCTCCCACCAGAAC,
NRP2-screen 1-R:
(SEQ ID NO: 22)
TTTCCTTGGGAGATTTGCTG,
NRP2-screen2-
F:
(SEQ ID NO: 23)
GAAATGATGTGTCCCCAACC,
NRP2-screen2-R:
(SEQ ID NO: 24)
AGAGCCGGAACAAGACAGAA).

To generate stable NRP2 knock-down cells, the following lentiviral shRNA vectors were used: TRCN0000063309. TRCN0000063310, shCtrl vectors is pLKO scramble shRNA (addgene, 1864). Lentivirus packaging vectors were obtained from Addgene pMD2.G (plasmid #12259) and psPAX2 (plasmid #12260). Plasmids were co-transfected into HEK-293T cells using Lipofectamine 3000 (Thermo Fisher Scientific, cat. L3000008). Virus was harvested 24 and 48 hrs after transfection, filtered through 0.45 μm filter, and added to the growth media of cells supplemented with 8 μg/ml polybrene (Sigma-Aldrich: #H9268). Stable cells were selected with 500 ng/ml puromycin for at least 2 weeks.

Lentiviral constructs were used to generate cells stably expressing NRP2-GFP⁵⁷, PD-L1-EGFP⁵⁸ and TAZ (4SA)⁴⁹. The lentiviral plasmid pGIPZ-PD-L1-EGFP (Addgene plasmid #120933) was a gift from Dr. Mien-Chic Hung⁵⁸. The NRP2-GFP plasmid was a gift from Dr. Cheng Chang. The pLVX-TAZ (4SA) plasmid was a gift from Dr. Bob Varelas. The virus production and transduction were performed as described above. Stable cells were selected with 500 ng/ml puromycin for at least 2 weeks.

RNA sequencing: RNA was extracted from the indicated cells and sent to Quick Biology for quantification, sequencing, and analysis. Each sample had a sequencing depth of 40 million. Data was analyzed at Bioinformatics Core in UMMS. Paired-end reads were aligned to human primary genome hg38, with star_2.5.3a, annotated with GENECODE GRCh38.p12 annotation release 29. Aligned exon fragments with mapping quality higher than 20 were counted toward gene expression with featureCounts 1.5.2. Differential expression (DE) analysis was performed with DESeq2_1.20.0. Within DE analysis, ‘ashr’ was used to create log 2 Fold Change (LFC) shrinkage for each comparison. Significant DE genes (DEGs) were filtered with the criteria FDR <0.05. Heatmaps were created with Prism. Gene set enrichment analysis were performed with GSEA.

Flow cytometry Cells were trypsinized, washed with PBS, and incubated with primary antibodies in complete medium for 1 hr at room temperature, followed by PBS washes and a 30 min incubation with secondary antibodies. Cells were washed 2 times in PBS and fixed with 2% paraformaldehyde in PBS and analyzed using BD LSR IIs flow cytometer (BD Biosciences) and FlowJo (BD).

Immunoblotting. Cells were washed PBS and scraped on ice in RIPA buffer with EDTA and EGTA (BP-115DG, Boston Bioproducts) supplemented with protease and phosphatase inhibitors (Roche, 04693132001). Laemmli buffer (BP-11IR. Boston Bioproducts) was added to each sample and the lysate was boiled and separated by SDS-PAGE.

Real-time qPCR. RNA extraction was accomplished using an RNA isolation kit (BS88133, Bio Basic Inc.), and cDNAs were produced using a qScript cDNA syn-thesis kit (#95047, Quantabio). SYBR Green (Applied Biosystems) was used as the qPCR master mix. Experiments were performed in triplicate and normalized to 18S. qPCR primer sequences were obtained from the Massachusetts General Hospital/Harvard Medical School PrimerBank (pga.mgh. harvard.edu/primerbank/).

Immunofluorescence microscopy. For fixed cell imaging, cells were cultured on coverslips, washed in PBS, and then fixed in 2% paraformaldehyde in PBS for 20 mins at room temperature. After fixation, cells were rinsed with PBS, permeabilized in PBS-0.5% Triton X-100 for 5 mins, blocked in blocking buffer (PBS, 5% Normal goat serum [Sigma], 0.1% Triton X-100, and 2 mM NaN₃) for 30 mins, and then incubated with primary antibodies in blocking buffer for 1 hr, at room temperature. Cells were washed 3 times, 5 mins each, in PBS-0.1% Triton X-100, then incubated with secondary antibodies and Hoechst 33342 (1:1,000; Invitrogen) for 1 hr, at room temperature. Cells were washed 3 times, 5 mins each, in PBS-0.1% Triton X-100, and mounted in 0.1M n-propyl gallate, 90% (by volume) glycerol, and 10% PBS solution.

In Vitro T cell assay. Human blood from healthy volunteers was collected into 150 ml Heparin-anticoagulated tubes and the mononuclear cells were collected by Ficoll density gradient centrifugation overlay method with 5 hours of collection of blood. Whole blood was mixed with an equal volume of RMPI-1640 (Gibco, Thermo fisher Scientific, USA) and the peripheral blood mononuclear cells were isolated by centrifugation at 1200 rpm for 30 mins at room temperature through Ficoll-Hypaque (Catalog #H889-500 ml, Sigma). The isolated buffy coat containing the mononuclear cells was washed twice with RPMI-1640 and the cell count was determined by trypan blue exclusion method using a hemocytometer. Blood was collected from the healthy volunteers after informed consent was obtained and all procedures related to human samples were performed according to the guidelines of Institute Biosafety Committee (IBC) of University of Massachusetts Medical School.

Isolated human PBMC were stained with Cell trace violet (Invitrogen, USA) at a final concentration of 10 μM for 8 mins at 37° C., water bath with intermittent shaking, and six times the volume of complete RPMI-1640 medium containing 10% FBS (Sigma-Aldrich) was added to stop the reaction. The stained cells were washed twice to remove excess Cell trace violet and the staining was further confirmed on a BD Accuri C6 Flow Cytometer (BD Biosciences, USA). The stained PBMC were co-cultured with PCa cells in complete RMPI-1640 medium containing 10% FBS in a round bottom 96-well plate for 5 days at different Suppressor: PBMC ratio (2:1, 1:1, 0.5:1 and 0.25:1). Duplicate wells were set-up for each Suppressor: PMC ratio, and 50 μl of culture medium was gently removed from the top of the well for cytokine analysis. Appropriate positive and negative controls were included; positive controls were the stained PBMC activated with T cell activator CD3/CD28 beads beads (Gibco, Thermo Fisher Scientific, USA), and PBMC only and PCa cells only wells were the two negative controls. To evaluate the proliferation of T cell lymphocytes in the presence of knockdown and wild type PC3 cells, on day 5 the cells were harvested and stained with the following antibodies: CD45, CD3, CD8, and CD4 (Clone: RPA-T4, BD BioSciences). A Fixable Viability Dye was also included in the FACS panel to exclude the dead cells from the analysis. Staining was followed by fixation with 4% paraformaldehyde and the samples were read on Aurora (Cytek Biosciences) using SpectroFlo software (Cytek Biosciences). At least, 100,000 events on lymphocyte gate were recorded and data was analyzed using FlowJo v10 software (TreeStar, Ashland, OR). Proliferation was determined based on the percentage of CFSE low cells.

Animal studies. For humanized mouse model experiments, 2 million prostate cancer cells were implanted subcutaneously in NOD.Cg-Prkdescidll2rgtm1Wjl (NSG) mice. Tumor onset was determined by palpation and used as time point to inject PBMCs. 20 million PBMCs were injected intravenously. Tumor volume was measured twice a week. For xenograft experiments involving drug treatment: NSG mice or C57BL/6J mice bearing TRAMP-C2 xenografts were treated with either control IgG or a-NRP2-28 (10 mg/kg, intraperitoneally, twice weekly). Tumor volume was measured twice as week.

Animal studies. For xenograft experiments involving drug treatment: NSG mice (NOD.Cg-Prkde^{scid}Il2rg^{tmlWjl}/SzJ from the Jackson Laboratory, stock #5557) or C57BL/6J mice (Jackson Laboratory, stock #664) bearing TRAMP-C2 xenografts were treated with either IgG control, aPD-L1 (10F.9G2, InVivoMAb Antibodies, 10 mg/kg, intraperitoncally, twice weekly), or aNRP2-28 (25 mg/kg, intraperitoneally, twice weekly). To initiate the tumor model, 2×10⁶ TRAMP-C2 cells suspended in 100 μl serum free media and Matrigel mixture (1:1) were used for each injection. Tumors were injected subcutaneously. Tumor onset was determined by palpation. Tumor size was measured by caliper two times a week and volume (mm³) were calculated by (length×width)²/2. A tumor size of 100 mm³ was used as the time point to initiate antibody treatment. All animal use was in accordance with the guidelines of the Animal Care and Use Committee of the University of Massachusetts Chan Medical School and conformed to the recommendations in the Guide for the Care and Use of Laboratory Animals (Institute of Laboratory Animal Resources, National Research Council. National Academy of Sciences, 1996).

Statistical analysis. The statistical significance of differences in average measurements was evaluated using unpaired t-tests (GraphPad Prism 9.0). Means are taken to be significantly different if P<0.05. In figures, “*” indicates 0.05>P≥0.01, “***” indicates 0.01>P≥0.001. “**** indicates 0.001>P≥0.0001,” ****** indicates 0.0001>P, and not significant “ns” indicates P≥0.05 for the indicated pairwise comparison. Error bars in all figures indicate standard deviation (SD).

### Example 1. NRP2 VEGF Signaling Promotes PD-L1 Transcription and Surface Expression

Experiments showed that the NRP2 and VEGF-A genes are significantly amplified in mCRPC and NEPC patient samples (FIG. 1A), which prompted us to investigate the role of NRP2 in mediating aggressive behaviors in lethal PCa using an unbiased approach, we sorted PC3 cells, an aggressive and metastatic PC cell line 36,37 based on NRP2 surface expression into a NRP2-high-expressing population (NRP2^{high}) and a NRP2-low-expressing population (NRP2^{low}) (FIG. 1B) and performed RNA-sequencing analysis (FIG. 1C, D). Gene set enrichment analysis (GSEA) using Hallmark gene set ^{b 38} showed that the 50 different gene sets were significantly enriched in NRP2^{High} population, including ones that have been reported to associate with NRP2 function, such as epithelial-mesenchymal-transition (EMT) ³⁹, Hypoxia ⁴⁰, angiogenesis⁴¹, and PI3K-AKT-mTOR signaling ⁴² (FIG. 1C). Interestingly, gene networks that are associated with immune response were significantly enriched in NRP2^{High} population, such as TNF-α/NF-κB pathway, IL2/STATS signaling, inflammatory response, IL6/JAK-STAT3 signaling, TGF-β signaling, and IFN-γ response (FIG. 1C). Importantly, all these gene networks can induce PD-L1 expression ⁴³. Indeed, our data revealed that NRP2^{High} populations expressed significantly higher PD-L1 comparing to NRP2^{Low} population (FIG. 1D) This result was further confirmed using qPCR to specifically quantify PD-L1 mRNA level in NRP2^{High} and NRP2^{Low} population in two different prostate cancer cell lines (PC3 and RWPE-2) (FIG. 1E). In both cell lines, the NRP2^{High} population expressed significantly more PD-L1 mRNA comparing to NRP2^{Low} population (FIG. 1E). Thus, our data suggest that NRP2 expression positively correlate with PD-L1 expression.

It has been reported that the NRP2 isoform NRP2b expression correlates with PD-L1 expression and poor survival in lung cancer patient samples⁴⁴. However, it is not known if NRP2 regulates PD-L1 expression. To investigate this, we used CRISPR/Cas9 to knock out NRP2 in PC3 cells (PC3^{NRP2KO}) (FIGS. 8A, B). We used two gRNAs to generate homozygous deletion of NRP2 exon2, which results in a frameshift leading to premature stop codons within the open reading frame (ORF) of NRP2. PCR screen confirmed the homozygous knockout of NRP2 exon 2 in the PC3^{NRP2KO} clone (FIG. 8B). To control for clonal effect, we also generated NRP2 depletion cells using two different shRNA against NRP2 (PC3-shNRP2). PC3 cells expressing scramble shRNA was used as control (PC3-shCtrl) (FIG. 8C). Knockdown of NRP2 using either CRISPR or shRNA significantly reduced PD-L1 mRNA comparing to control cells (FIG. 2A and FIG. 8D). Consequently. PD-L1 total protein expression and cell surface expression were eliminated in cells with NRP2 depletion (FIGS. 2B, C, and D). To investigate if the effect of NRP2 on PD-L1 expression is mediated by VEGF/NRP2 signaling, we knocked down VEGF-A or VEGF-C, the ligands of NRP2, using siRNA (FIG. 8D) and observed that depletion of either VEGF-A or VEGF-C significantly reduced PD-L1 surface expression (FIG. 2F). To further validate that the loss of PD-L1 expression was specifically due to NRP2 depletion, we re-expressed NRP2 tagged with GFP in PC3-shNRP2 cells (PC3-shNRP2-NRP2-GFP) (FIGS. 2F and G and FIG. 8E). This rescued PD-L1 mRNA and cell surface expression (FIGS. 2F, G). To investigate if the regulation of PD-L1 expression through NRP2 is conserved in different model system and subtypes of aggressive PCa, we used a mouse NEPC cell line TRAMP-C2 45 and used shRNA to knockdown NRP2 in these cells (FIG. 2H). Consistent with human metastatic PCa cell lines, knockdown of NRP2 in TRAMP-C2 cells reduced their PD-L1 surface expression comparing to control cells (FIG. 2H). Together, our data revealed a novel role of NRP2/VEGF signaling in regulating PD-L1 expression in aggressive PCa cells.

### Example 2. NRP 2 Promotes PD-L1 Transcription Through Activation of Hippo Pathway

We previously reported that NRP2 contributes to the Hippo effector TAZ activation in breast cancer cells ⁴⁶. In human breast cancer cells, TAZ promotes PD-L1 transcription ⁴⁷. Thus, we sought to determine if NRP2 regulates PD-L1 transcription in PCa through activation of Hippo pathway transducer YAP/TAZ. NRP2 knockdown in PCa PC3 cells significantly reduced the expression of TAZ target gene CTGF and CYR61 (FIG. 3A). Fluorescent microscopy imaging analysis showed that the percentage of cells with nuclear YAP and TAZ were significantly reduced in NRP2 knockout PC3 cells (PC3NRP2KO) comparing to NRP2 wild-type cells. (FIGS. 3B, C). YAP inhibition with verteporfin ⁴⁸ significantly reduced PD-L1 mRNA level (FIG. 3D). To investigate if TAZ activation promotes PD-L1 transcription downstream of NRP2, we used constitutively activated TAZ (TAZ (4SA)), which has all four serine residues in the HXRXXS motif replaced with alanine and is resistant to phosphorylation ⁴⁹, and expressed it in PC3^{NRP2KO} cells (FIG. 3E). Expression of TAZ (4SA) rescued PD-L1 mRNA expression in PC3NRP2KO cells (FIG. 3E). Further, expression of TAZ (4SA) increased PD-L1 surface expression in PC3^{NRP2KO} cells and PC3-shNRP2 cells (FIG. 3F). Together, our data suggest that NRP2 contributes to PD-L1 expression by promoting YAP/TAZ activation.

### Example 3. NRP2 in Tumor Cells Inhibits T Cell Activation In Vitro

We next sought to determine if NRP2 inhibits T cell function by promoting PD-L1 expression. To do this, we co-cultured activated T cells from primary peripheral blood mononuclear cells (PBMCs) with PC3-shCtrl, PC3-shNRP2, PC3-shNRP2-NRP2-GFP, or PC3-shNRP2 with re-expression of PD-L1 tagged with GFP (PC3-shNRP2-PD-L1-EGFP) (FIG. 4A, FIG. 8E, F). To specifically investigate T-cell suppression independently of tumor cell mediated T cell activation such as MHC loss on tumor cells or insufficient tumor associated antigens, we incubated PBMCs with anti-CD3/CD28 beads to activate T cells independent of tumor cells (FIG. 4A). T cells were pre-labeled with proliferation dye and the percentage of proliferating cells were used as a readout for activated T cells⁵⁰. After one week of incubation, we analyzed the cells by flow cytometry to quantify the percentage of proliferating CD4⁺ or CD8⁺ T cells (FIGS. 4A, B). When PCa cells were co-cultured with PBMCs at a ratio of 0.25:1, about 30% CD4⁺T cells or CD8⁺T cells cultured with PC3-shCtrl cells were proliferative (FIGS. 4B, C). By striking comparison, when PBMCs were co-cultured with PC3-shNRP2 cells, significantly more proliferating CD4⁺ T cells and CD8⁺ T cells were detected (FIGS. 3B, C). Interestingly, all the T cells co-cultured with PC3-shNRP2-NRP2-GFP or PC3-shNRP2-PD-L1-EGFP cells were dead regardless of the ratio between tumor cells versus PBMCs (FIG. 9). This is potentially due to the high amount of PD-L1 expressed on these cells that resulted in T cell apoptosis ⁵¹. Cytokine analysis revealed that IFN-g and pro-inflammatory cytokine interleukin (IL)-17A (IL-17A), cytokines that can be secreted by CD4+ T cells and CD8+ T cells ^{52,53}, were significantly enriched in superatant from PBMCs co-cultured with PC3-shNRP2 cells or activation beads only than supernatant from PBMCs co-cultured with PC3-shCtrl cells (FIG. 4D). Expression of NRP2 or PD-L1 in PC3-shNRP2 cells inhibited both IFN-g and IL-17A production from PBMCs co-cultured with them (FIG. 4D). Contrary to IFN-g or IL-17A, interleukin-2 (IL-2), a cytokine mainly produced by activated CD4+ T cells and is then consumed by cells that express the IL-2 receptor subunit CD25 like regulatory T (TReg) cells ⁵⁴, was significantly decreased in supernatant from PBMCs co-cultured with PC3-shNRP2 cells or activation beads only than PBMCs co-cultured with PC3-shCtrl cells (FIG. 4D). Expression of NRP2 or PD-L1 in PC3 cells rescued IL-2 production in PBMCs co-cultured with them (FIG. 4D). Since re-expression of PD-L1 in PC3-shNRP2 cells resulted in the same results as re-expression of NRP2 in PC3-shNRP2 cells, it suggests that the T cell inhibition mediated by NRP2 is potentially through PD-L1. Together, our data suggest that NRP2 in PCa cells inhibits T cell activation through PD-L1.

### Example 4. NRP2 Loss Results in Immune Mediated Tumor Elimination In Vivo

To investigate the effect of NRP2 on anti-tumor immunity in more closely recapitulate human biological systems in vivo, we used a humanized mouse model generated by engrafting human peripheral blood mononuclear cells (PBMC) intravenously (IV) into human PCa bearing NSG KbDb ABI mice that lack MHC class II molecules ⁵⁵ (FIG. 5A). This mouse model generates a functional human immune system with predominantly T cell engraftment with reduced xenogeneic graft-versus-host disease (GVHD) ^{55,56}. In NSG KbDb ABI mice that lack PBMCs engraftment, both PC-shCtrl cells and PC3-shNRP2 cells started forming detectable tumors around day 30 following tumor cell injection (FIG. 10A). In both groups, all the mice developed detectable tumors around Day 42 following tumor cell injection (FIG. 10A) with slower tumor onset observed in mice injected with PC3-shNRP2 cells (FIG. 10A). This showed that NRP2 Joss alone has modest impact on prostate tumorigenesis when implanted subcutaneously in immune compromised mice ²⁷. To specifically assess the impact of NRP2 depletion on anti-tumor immunity, with two independent experiments, we engrafted PBMCs from two healthy male donors into the mice after detectable tumors were established (FIGS. 5A, B). To assess the degree of immune cell development in the peripheral blood following PBMC injection, we collected blood from mice at 2 weeks and 4 weeks following PBMC engraftment and immune cell populations were analyzed by flow cytometry (FIGS. 10B, C). In mice injected with either PC3-shCtrl or PC3-shNRP2 cells, following 2 weeks of PBMC injection, both groups showed about 5% hCD45⁺ cells in peripheral blood, and it went up to about 30% after 4 weeks of PBMC injection. About 90% of CD45⁺ cells were CD3+ T cells (FIGS. 10B, C), validating the predominantly T cell engraftment in this mouse model. Interestingly, the percentage of CD8*/CD4⁺T cell ratio in CD3+ cells increased from 0.43 (30% CD8⁺T cells. 70% CD4⁺T cells) at 2 weeks after PBMC injection to 2.3 (70% CD8⁺T cells, 30% CD4⁺T cells) (FIGS. 10B, C), indicating an increase of activated cytotoxicity T cells that can function in anti-tumor immunity. Interestingly, in mice injected with control PCa cells that express scramble shRNA (PC3-shCtrl), engrafting PBMCs resulted in tumor regression starting two weeks following engraftment in the both sets of experiment (FIG. 4B). Residual tumors formed from PC3-shCtrl cells in mice injected with PBMCs displayed hCD45+immune cell infiltration (FIG. 10D). This suggests that PC3-shCtrl cells can be infiltrated with immune cells and can partially activate the immune system to suppress tumor growth. T cell infiltration were also detected in these tumors with 75% CD8⁺T cells and 25% CD4⁺ T cells (FIG. 10D), suggesting that the human T cells might be impeding tumor growth. Strikingly, engrafting PBMCs resulted in complete tumor elimination in 75% mice injected with NRP2-depleted cells (PC3-shNRP2) 2 weeks after PBMCs engrafting while PC3-shNRP2 cells injected in NSG mice without PBMC's engraftment continued to grow (FIG. 4D). Thus, our data indicating that NRP2 loss results in immune mediated tumor elimination in vivo.

Our data suggest that targeting NRP2 is a promising therapeutic strategy to activate anti-tumor immunity in treating cancers that currently have low response rate to PD blockade. Importantly. NEPC has not been amenable to immunotherapy, especially PD pathway blockade for multiple reasons including the observation that PD-L1 is present in exosomes secreted by cells and that exosome-localized PD-L1 is resistant to such blockade ²². This study concluded that PD-L1 expression itself must be suppressed to overcome PD-L1-mediated immune evasion. Given that it is difficult, if not impossible, to target PD-L1 expression directly with existing reagents, the approach of targeting VEGF/NRP2 to diminish PD-L1 expression is not only plausible but supported strongly by our observation that treatment with a NRP2 functional blocking mAb (aNRP2-28, a highly-specific mouse surrogate mAb that blocks the binding of VEGF to murine NRP2, developed by aTyr Pharma was used in these animal studies because it specifically inhibits the binding of VEGF to NRP2 (39), through a similar epitope, and with similar functional characteristics as aNRP2-10, a human therapeutic candidate antibody) reduced PD-L1 expression in TRAMP-C2 cells (FIG. 6A). To test the therapeutic benefit of a-NRP2 mAb in vivo, we engrafted TRAMP-C2 cells to C57BL/6J mice, a syngeneic model of NEPC that is resisted to PD-1/PD-L1 blockade 21 (FIG. 6B). We treated mice with established tumors with either IgG control or a-NRP2 mAb (FIG. 6B). The results observed were striking. Treatment with aNRP2-28, given as monotherapy, significantly reduced tumor burden (FIG. 6C), while treatment with the aPD-L1 mAb did not have an impact on tumor volume compared to IgG control (FIG. 11A). The inhibition of tumor growth was specifically due to anti-tumor immunity because no difference was observed in NSG mice treated with either IgG control or a-NRP2 mAb (FIGS. 6B, C). Importantly, a-NRP2 mAb had a profound impact on diminishing PD-L1 expression in tumors in vivo (FIGS. 6D, E). Tumors treated with aNRP2-28 showed a significant increase in tumor infiltrating immune cells, including activated effector cells such as granzyme B expressing CD8⁺ T cells and NK cells (FIGS. 7A, B). Taken together, the data provide strong support for targeted NRP2 inhibition to activate anti-tumor immunity in lethal PCa.

Tumor volume is only one parameter for assessing the effect of therapy and it can be influenced by necrosis and immune cell infiltration. For this reason, we analyzed tumor histology to gain additional insight into the response to aNRP2-28 and aPD-L1 therapy. Strikingly, aNRP2-28 treatment caused a significant increase in necrosis in tumor-bearing C57BL/6J mice compared to IgG treatment (p=0.0260);

FIG. 11B. However, treatment with the aPD-L1 mAb did not cause a significant increase in necrosis (FIG. 11C, p=0.6572). This observation is important because tumor necrosis is an important criterion in evaluating response to therapy. No significant difference in tumor necrosis was observed in immune compromised NSG mice (p=0.7846), suggesting the increased tumor necrosis with aNRP2-28 treatment was specifically due to anti-tumor immunity.

Importantly, aNRP2-28 treatment of TRAMP-C2 xenografts in syngeneic mice diminished PD-L1 expression in the residual non-necrotic regions of tumors significantly compared to the IgG control (p=0.0007). Further assessment of the tumor microenvironment using quantitative flow cytometry revealed that aNRP2-28 treatment caused an increase in tumor-infiltrating immune cells, including CD4⁺ and CD8⁺T cells, compared to control. In contrast, an aPD-L1 mAb did not have an effect on these cells. Importantly, activated effector cells (CD69′ CD3+ T cells, granzyme B-expressing CD8⁺T cells, and granzyme B-expressing NK cells) were also significantly increased in response to aNRP2-28 compared to IgG treatment (p=0.0002, p=0.0131, p=0.0011, respectively.

We next sought to investigate a correlation between VEGF/NRP2 and PD-L1 expression in aggressive subtypes of prostate cancer including metastatic castration resistant prostate cancer (mCRPC) and NEPC. Further analysis of published prostate cancer datasets in cBioPortal^{60,61} revealed that the NRP2, VEGFA, and VEGFC genes are amplified in mCRPC and NEPC patient samples (FIG. 12A). Also, there was significant co-occurrence (p-Value <0.001; q-Value, <0.001) between NRP2 and VEGFC gene amplification among the patient samples. Analysis of published prostate cancer datasets (SU2C/PCF Dream Team) ⁶² revealed a significant positive correlation between NRP2 and CD274 mRNA expression in metastatic prostate patient samples (p<0.0001) (FIG. 12B). Our analysis revealed that the NRP2^{high} PD-L 1high population of cells in patients with metastatic tumors⁶² showed significantly lower androgen receptor (AR) and higher NEPC scores than NRP2^{low} PD-L1 ^{low} population in a published prostate cancer dataset⁶² (p<0.0001, p=0.0070, for AR and NEPC scores, respectively) (FIG. 12C). These data prompted us to investigate the therapeutic benefit of inhibiting the binding of VEGF to NRP2 using human NEPC models. For this purpose, we used a characterized NEPC patient-derived organoid model OWCM126263. The organoid lacks AR protein expression and expresses classical neuroendocrine markers⁶³. Importantly, we observed that this organoid model expresses surface NRP2 and PD-L1 (FIG. 12D). This observation is consistent with other studies, which reported that PD-L1 expression increases in advanced prostate cancers^{11, 12, 64, 65} and, more specifically, that it is expressed in 41% of NEPC⁶⁵. To investigate whether inhibiting the binding of VEGF to NRP2 decreases PD-L1 expression, organoids were treated with either IgG control or aNRP2 mAb (aNRP2-10), a humanized functional mAb developed by aTyr Pharma that specifically blocks the binding of VEGF to NRP2 targeting the same epitope as murine aNRP2-28⁵⁹. Treatment with aNRP2-10, but not control IgG, significantly reduced PD-L1 surface expression in this organoid system (p=0.0048) (FIG. 12E). These findings demonstrate that VEGF/NRP2 signaling sustains PD-L1 expression in a patient-derived organoid model of NEPC and that PD-L1 expression can be diminished by mAb-mediated inhibition of VEGF binding to NRP2.

The above findings prompted us to investigate the effect of aNRP2-10 on immune-mediated tumor cell killing using a tumor organoid-T-cell coculture system⁶⁶ that involves co-culturing organoids with activated hPBMCs. Using this system, we observed that aNRP2-10 treatment resulted in a significant increase in tumor cell death compared to IgG control treatment (p=0.0276) (FIGS. 12F and G). No difference in viability was observed in organoids treated with either IgG control or aNRP2-10 without activated PBMCs (FIGS. 12F and G), indicating that the cell death observed was caused by immune cell mediated-killing.

### REFERENCES

1 Huggins, C. & Hodges, C. V. Studies on prostatic cancer: I. The effect of castration, of estrogen and of androgen injection on serum phosphatases in metastatic carcinoma of the prostate. 1941. J Urol 168, 9-12, doi: 10.1016/s0022-5347 (05) 64820-3 (2002).
2 Lee, J. K, et al. Systemic surfaceome profiling identifies target antigens for immune-based therapy in subtypes of advanced prostate cancer. Proc Natl Acad Sci USA 115, E4473-E4482, doi: 10.1073/pnas. 1802354115 (2018).
3 Robinson, D, et al. Integrative Clinical Genomics of Advanced Prostate Cancer. Cell 162, 454, doi: 10.1016/j.cell.2015.06.053 (2015).
4 Rickman, D. S., Beltran, H., Demichelis, F. & Rubin, M. A. Biology and evolution of poorly differentiated neuroendocrine tumors. Nat Med 23, 1-10, doi: 10.1038/nm.4341 (2017).
5 Beltran, H, et al. Aggressive variants of castration-resistant prostate cancer. Clin Cancer Res 20, 2846-2850, doi: 10.1158/1078-0432.CCR-13-3309 (2014).
6 Beltran, H, et al. Molecular characterization of neuroendocrine prostate cancer and identification of new drug targets. Cancer Discov 1, 487-495. doi: 10.1158/2159-8290.CD-11-0130 (2011).
7 Beltran, H, et al. The Role of Lineage Plasticity in Prostate Cancer Therapy Resistance. Clin Cancer Res 25, 6916-6924, doi: 10.1158/1078-0432.CCR-19-1423 (2019).
8 Miller, A. M. & Pisa, P. Tumor escape mechanisms in prostate cancer. Cancer Immunol Immunother 56, 81-87, doi: 10.1007/s00262-005-0110-x (2007).
9 Wu, Y. M, et al. Inactivation of CDK12 Delineates a Distinct Immunogenic Class of Advanced Prostate Cancer. Cell 173, 1770-1782 e1714, doi: 10.1016/j.cell.2018.04.034 (2018).
10 Strasner, A. & Karin, M. Immune Infiltration and Prostate Cancer. Front Oncol 5, 128, doi: 10.3389/fonc.2015.00128 (2015).
11 Bishop, J. L, et al. PD-L1 is highly expressed in Enzalutamide resistant prostate cancer. Oncotarget 6, 234-242, doi: 10.18632/oncotarget.2703 (2015).
12 Gevensleben, H, et al. The Immune Checkpoint Regulator PD-L1 Is Highly Expressed in Aggressive Primary Prostate Cancer. Clin Cancer Res 22, 1969-1977, doi: 10.1158/1078-0432.CCR-15-2042 (2016).
13 Hamid, O, et al. Safety and tumor responses with lambrolizumab (anti-PD-1) in melanoma. N Engl J Med 369, 134-144, doi: 10.1056/NEJMoal305133 (2013).
14 Powles, T, et al. MPDL3280A (anti-PD-L1) treatment leads to clinical activity in metastatic bladder cancer. Nature 515, 558-562, doi: 10.1038/nature 13904 (2014).
15 Le, D. T, et al. PD-1 Blockade in Tumors with Mismatch-Repair Deficiency. N Engl J Med 372, 2509-2520, doi: 10.1056/NEJMoa1500596 (2015).
16 Herbst. R. S, et al. Pembrolizumab versus docetaxel for previously treated, PD-L1-positive, advanced non-small-cell lung cancer (KEYNOTE-010): a randomised controlled trial. Lancet 387, 1540-1550, doi: 10.1016/S0140-6736 (15) 01281-7 (2016).
17 Topalian, S. L, et al. Safety, activity, and immune correlates of anti-PD-1 antibody in cancer. N Engl J Med 366, 2443-2454, doi: 10.1056/NEJMoa1200690 (2012).
18 Graff, J. N, et al. Early evidence of anti-PD-1 activity in enzalutamide-resistant prostate cancer. Oncotarget 7, 52810-52817, doi: 10.18632/oncotarget. 10547 (2016).
19 Hansen, A. R, et al. Pembrolizumab for advanced prostate adenocarcinoma: findings of the KEYNOTE-028 study. Ann Oncol 29, 1807-1813, doi: 10.1093/annonc/mdy²³² (2018).
Isaacsson Velho, P. & Antonarakis, E. S. PD-1/PD-L1 pathway inhibitors in advanced prostate cancer. Expert Rev Clin Pharmacol 11, 475-486, doi: 10.1080/17512433.2018.1464388 (2018).
21 Yu, P, et al. Simultaneous inhibition of two regulatory T-cell subsets enhanced Interleukin-15 efficacy in a prostate tumor model. Proc Natl Acad Sci U S A 109, 6187-6192, doi: 10.1073/pnas. 1203479109 (2012).
22 Poggio, M, et al. Suppression of Exosomal PD-L1 Induces Systemic Anti-tumor Immunity and Memory. Cell 177, 414-427 e413, doi: 10.1016/j.cell.2019.02.016 (2019).
23 Kolodkin, A. L, et al. Neuropilin is a semaphorin III receptor. Cell 90, 753-762, doi: 10.1016/s0092-8674 (00) 80535-8 (1997).
24 Chen, H., Chedotal, A., He, Z., Goodman, C. S. & Tessier-Lavigne, M. Neuropilin-2, a novel member of the neuropilin family, is a high affinity receptor for the semaphorins Sema E and Sema IV but not Sema III. Neuron 19, 547-559. doi: 10.1016/s0896-6273 (00) 80371-2 (1997).
25 Soker, S., Takashima, S., Miao, H. Q., Neufeld, G. & Klagsbrun, M. Neuropilin-1 is expressed by endothelial and tumor cells as an isoform-specific receptor for vascular endothelial growth factor. Cell 92, 735-745, doi: 10.1016/s0092-8674 (00) 81402-6 (1998).
26 Roy, S, et al. Multifaceted Role of Neuropilins in the Immune System: Potential Targets for Immunotherapy. Front Immunol 8, 1228, doi: 10.3389/fimmu.2017.01228 (2017).
27 Goel, H. L, et al. VEGF/neuropilin-2 regulation of Bmi-1 and consequent repression of IGF-IR define a novel mechanism of aggressive prostate cancer. Cancer Discov 2, 906-921, doi: 10.1158/2159-8290.CD-12-0085 (2012).
28 Goel, H. L. & Mercurio, A. M. VEGF targets the tumour cell. Nat Rev Cancer 13, 871-882, doi: 10.1038/nrc3627 (2013).
29 Goel, H. L, et al. P-Rex l Promotes Resistance to VEGF/VEGFR-Targeted Therapy in Prostate Cancer. Cell Rep 14, 2193-2208, doi: 10.1016/j.celrep.2016.02.016 (2016).
30 Mercurio, A. M. VEGF/Neuropilin Signaling in Cancer Stem Cells. Int J Mol Sci 20, doi: 10.3390/ijms20030490 (2019).
31 Elaimy, A. L, et al. VEGF-neuropilin-2 signaling promotes stem-like traits in breast cancer cells by TAZ-mediated repression of the Rac GAP beta2-chimacrin. Sci Signal 11, doi: 10.1126/scisignal aao6897 (2018).
32 McMenamin, M. E, et al. Loss of PTEN expression in paraffin-embedded primary prostate cancer correlates with high Gleason score and advanced stage. Cancer Res 59, 4291-4296 (1999).
33 Carver, B. S, et al. Aberrant ERG expression cooperates with loss of PTEN to promote cancer progression in the prostate. Nat Genet 41, 619-624, doi: 10.1038/ng.370 (2009).
34 Zou, W., Wolchok, J. D. & Chen, L. PD-L1 (B7-H1) and PD-1 pathway blockade for cancer therapy: Mechanisms, response biomarkers, and combinations. Sci Transl Med 8, 328rv324, doi: 10.1126/scitranslmed.aad7118 (2016).
35 Caunt, M, et al. Blocking neuropilin-2 function inhibits tumor cell metastasis. Cancer Cell 13. 331-342, doi: 10.1016/j.ccr.2008.01.029 (2008).
36 Cunningham, D. & You, Z. In vitro and in vivo model systems used in prostate cancer research. J Biol Methods 2, doi: 10.14440/jbm.2015.63 (2015).
37 Liu. Q, et al. Interleukin-Ibeta promotes skeletal colonization and progression of metastatic prostate cancer cells with neuroendocrine features. Cancer Res 73, 3297-3305, doi: 10.1158/0008-5472.CAN-12-3970 (2013).
38 Liberzon, A, et al. The Molecular Signatures Database (MSigDB) ballmark gene set collection. Cell Syst 1, 417-425, doi: 10.1016/j.cels.2015.12.004 (2015).
39 Kong, D, et al. VEGF-C mediates tumor growth and metastasis through promoting EMT-epithelial breast cancer cell crosstalk. Oncogene 40, 964-979, doi: 10.1038/s41388-020-01539-x (2021).
40 Mukhopadhyay, D. & Datta, K. Multiple regulatory pathways of vascular permeability factor/vascular endothelial growth factor (VPF/VEGF) expression in tumors. Semin Cancer Biol 14, 123-130, doi: 10.1016/j.semcancer.2003.09.019 (2004).
41 Alghamdi, A. A. A, et al. NRP2 as an Emerging Angiogenic Player; Promoting Endothelial Cell Adhesion and Migration by Regulating Recycling of alpha5 Integrin. Front Cell Dev Biol 8, 395, doi: 10.3389/fcell.2020.00395 (2020).
42 Gray, M. J, et al. Therapeutic targeting of neuropilin-2 on colorectal carcinoma cells implanted in the murine liver. J Natl Cancer Inst 100, 109-120, doi: 10.1093/jnci/djm279 (2008).
43 Sun, C., Mezzadra, R. & Schumacher, T. N. Regulation and Function of the PD-L1 Checkpoint. Immunity 48, 434-452, doi: 10.1016/j.immuni.2018.03.014 (2018).
44 Gemmill. R. M, ct al. The neuropilin 2 isoform NRP2b uniquely supports TGFbeta-mediated progression in lung cancer. Sci Signal 10, doi: 10.1126/scisignal.aag0528 (2017).
45 Foster, B. A., Gingrich, J. R., Kwon, E. D., Madias, C. & Greenberg, N. M. Characterization of prostatic epithelial cell lines derived from transgenic adenocarcinoma of the mouse prostate (TRAMP) model. Cancer Res 57, 3325-3330 (1997).
46 Wang, M., Knudsen, B. S., Nagle, R. B., Rogers, G. C. & Cress, A. E. A method of quantifying centrosomes at the single-cell level in human normal and cancer tissue. Mol Biol Cell 30, 811-819, doi: 10.1091/mbc.E18-10-0651 (2019).
47 Janse van Rensburg, H. J, et al. The Hippo Pathway Component TAZ Promotes Immune Evasion in Human Cancer through PD-L1. Cancer Res 78, 1457-1470, doi: 10.1158/0008-5472.CAN-17-3139 (2018).
48 Liu-Chittenden, Y, et al. Genetic and pharmacological disruption of the TEAD-YAP complex suppresses the oncogenic activity of YAP. Genes Dev 26. 1300-1305, doi: 10.1101/gad.192856.112 (2012).
49 Lei, Q. Y, et al. TAZ promotes cell proliferation and epithelial-mesenchymal transition and is inhibited by the hippo pathway. Mol Cell Biol 28, 2426-2436, doi: 10.1128/MCB.01874-07 (2008).
50 Smith-Garvin, J. E., Koretzky, G. A. & Jordan, M. S. T cell activation. Annu Rev Immunol 27, 591-619, doi: 10.1146/annurev.immunol.021908.132706 (2009).
51 Shi, F, et al. PD-1 and PD-L1 upregulation promotes CD8 (+) T-cell apoptosis and postoperative recurrence in hepatocellular carcinoma patients. Int J Cancer 128. 887-896, doi: 10.1002/ijc.25397 (2011).
52 Farrar, M. A. & Schreiber, R. D. The molecular cell biology of interferon-gamma and its receptor. Annu Rev Immunol 11, 571-611, doi: 10.1146/annurev.iy.11.040193.003035 (1993).
53 Srenathan, U., Steel, K. & Taams, L. S. IL-17+CD8⁺T cells: Differentiation, phenotype and role in inflammatory disease. Immunol Lett 178, 20-26, doi: 10.1016/j.imlet.2016.05.001 (2016).
54 Boyman, O. & Sprent, J. The role of interleukin-2 during homeostasis and activation of the immune system. Nat Rev Immunol 12, 180-190, doi: 10.1038/nri3156 (2012).
55 Covassin. L, et al. Human peripheral blood CD4 T cell-engrafted non-obese diabetic-scid IL2rgamma (null) H2-Abl (tm 1Gru) Tg (human leucocyte antigen D-related 4) mice: a mouse model of human allogeneic graft-versus-host disease. Clin Exp Immunol 166, 269-280, doi: 10.1111/j.1365-2249.2011.04462.x (2011).
56 Shultz, L. D., Brehm, M. A., Garcia-Martinez, J. V. & Greiner, D. L. Humanized mice for immune system investigation: progress, promise and challenges. Nat Rev Immunol 12, 786-798, doi: 10.1038/nr3311 (2012).

57 Bac. D., Lu, S., Taglienti, C. A. & Mercurio, A. M. Metabolic stress induces the lysosomal degradation of neuropilin-1 but not neuropilin-2. J Biol Chem 283, 28074-28080, doi: 10.1074/jbc.M804203200 (2008).
58 Yang. Y, et al. Exosomal PD-L1 harbors active defense function to suppress T cell killing of breast cancer cells and promote tumor growth. Cell Res 28, 862-864, doi: 10.1038/s41422-018-0060-4 (2018).
59 Z. Xu et al., Abstract LB095: A domain-specific antibody to NRP2 down-regulated epithelial-mesenchymal transition genes and enhanced efficacy of standard-of-care therapeutics for aggressive breast cancer. Cancer Research 81. LB095-LB095 (2021): US20210163606; U.S. Pat. No. 11,505,610).
60. E. Cerami et al., The cBio cancer genomics portal: an open platform for exploring multidimensional cancer genomics data. Cancer Discov 2, 401-404 (2012).
61. J. Gao et al., Integrative analysis of complex cancer genomics and clinical profiles using the cBioPortal. Sci Signal 6, p 11 (2013).
62. W. Abida et al., Genomic correlates of clinical outcome in advanced prostate cancer. Proc Natl Acad Sci USA 116, 11428-11436 (2019).
63. L. Puca et al., Patient derived organoids to model rare prostate cancer phenotypes. Nat Commun 9, 2404 (2018).
64. M. C. Haffner et al., Comprehensive Evaluation of Programmed Death-Ligand I Expression in Primary and Metastatic Prostate Cancer. Am J Pathol 188, 1478-1485 (2018).
65. L. Nappi et al., Immunogenomic landscape of neuroendocrine small cell prostate cancer. Journal of Clinical Oncology 37, 217-217 (2019).
66. C. M. Cattaneo et al., Tumor organoid-T-cell coculture systems. Nat Protoc 15, 15-39 (2020).
67. H. Beltran et al., Divergent clonal evolution of castration-resistant neuroendocrine prostate cancer. Nat Med 22, 298-305 (2016).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject who has metastatic castration-resistant prostate cancer (mCRPC) or neuroendocrine prostate cancer (NEPC), the method comprising administering to the subject a therapeutically effective amount of an inhibitor of Neuropilin 2 (NRP2).

2. The method of claim 1, wherein the inhibitor is selected from the group consisting of antibodies, inhibitory peptides, small molecules, and inhibitory nucleic acids.

3. The method of claim 2, wherein the antibody is aNRP2-28, optionally aNRP2-28v2 or aNRP2-28v4, or aNRP2-10v10.

4. The method of claim 2, wherein the inhibitory peptide comprises soluble NRP2 B domain, optionally with mutations R287E and N290D.

5. The method of claim 2, wherein the small molecule is Zafirlukast, Actinomycin D, Dihydrexidine, or a benzamidine inhibitor of VEGF-C binding to NRP2.

6. The method of claim 2, wherein the inhibitory nucleic acids are antisense oligonucleotides (ASOs) or single- or double-stranded RNA interference (RNAi) compounds such as siRNA or shRNA that bind to a nucleic acid encoding NRP2.

7. (canceled)

8. The method of claim 1, further comprising administering a treatment comprising an anti-angiogenic agent.

9. The method of claim 8, wherein the anti-angiogenic agent is a VEGF inhibitor; aflibercept; or a Tyrosine kinase inhibitor (TKI).

10. (canceled)

11. A method for treating a neuroendocrine prostate cancer (NEPC) in a human subject in need thereof, comprising administering to the subject an antibody or an antigen-binding fragment thereof that specifically binds to a human neuropilin-2 (NRP2) polypeptide at an epitope in the neuropilin b1 domain of NRP2, thereby treating the NEPC in the subject in need thereof.

12. The method of claim 11, wherein the antibody or antigen-binding fragment thereof comprises:
a heavy chain variable region (V_{H}) sequence that comprises complementary determining region (CDR) V_{H}CDR1, V_{H}CDR2, and V_{H}CDR3 sequences set forth in SEQ ID NOs: 1-3, respectively, and a light chain variable region (V_{L}) sequence that comprises complementary determining region V_{L}CDR1, V_{L}CDR2, and V_{L}CDR3 sequences set forth in SEQ ID NOs: 4-6, respectively; or
a V_{H} sequence that comprises V_{H}CDR1, V_{H}CDR2, and V_{H}CDR3 sequences set forth in SEQ ID NOs: 7-9, respectively, and V_{L} sequence that comprises V_{L}CDR1, V_{L}CDR2, and V_{L}CDR3 sequences set forth in SEQ ID NOs: 10-12, respectively.

13. The method of claim 12, wherein:
the V_{H} sequence comprises a sequence at least 80, 85, 90, 95, 97, 98, 99, or 100% identical to SEQ ID NO: 13, and the V_{L} sequence comprises a sequence at least 80, 85, 90, 95, 97, 98, 99, or 100% identical to SEQ ID NO: 14;
the V_{H} sequence comprises a sequence at least 80, 85, 90, 95, 97, 98, 99, or 100% identical to SEQ ID NO: 15, and the V_{L} sequence comprises a sequence at least 80, 85, 90, 95, 97, 98, 99, or 100% identical to SEQ ID NO: 16; or
the V_{H} sequence comprises a sequence at least 80, 85, 90, 95, 97, 98, 99, or 100% identical to SEQ ID NO: 17, and the V_{L} sequence comprises a sequence at least 80, 85, 90, 95, 97, 98, 99, or 100% identical to SEQ ID NO: 18.

14. The method of claim 11, wherein the antibody or antigen-binding fragment thereof is a humanized monoclonal antibody that comprises a human IgG4 Fc domain.

15. The method of claim 11, wherein the NEPC is characterized by cell morphology/histology and/or expression of one or more NEPC markers.

16.-23. (canceled)

24. The method of claim 11, wherein the human subject has received at least 1 or 2 lines of systemic therapy for prostate cancer and has relapsed from the last systemic therapy.

25. The method of claim 24, wherein the last systemic therapy is selected from one or more of hormonal therapy via surgical or chemical castration (LHRH agonist), chemotherapy, and radiopharmaceutical therapy.

26. The method of claim 11, comprising administering the inhibitor of NRP2, optionally an antibody or antigen-binding fragment thereof, in combination with a treatment comprising an anti-angiogenic agent, or at least one or two additional chemotherapeutic agents.

27. The method of claim 26, wherein the at least one additional chemotherapeutic agent is selected from etoposide, carboplatin, cisplatin, and docetaxel; or wherein the anti-angiogenic agent is a VEGF inhibitor, optionally an antibody (optionally bevacizumab, ramucirumab, or ranibizumab); aflibercept; or a Tyrosine kinase inhibitor (TKI) (optionally sunitinib, pazopanib, sorafenib, nilotinib, axitinib, or dasatinib).

28. The method of claim 26, wherein the at least two additional chemotherapeutic agents are selected from etoposide+carboplatin, etoposide+cisplatin, and docetaxel+carboplatin.

29.-52. (canceled)

53. A patient care kit, comprising:
means for determining the presence or absence of a neuroendocrine prostate cancer (NEPC) in a tissue sample from a human patient with prostate cancer; and
an antibody or an antigen-binding fragment thereof that specifically binds to a human neuropilin-2 (NRP2) polypeptide at an epitope in the neuropilin b1 domain of NRP2.

54. The patient care kit of claim 53, wherein the antibody or antigen-binding fragment thereof of (b) comprises:
a heavy chain variable region (V_{H}) sequence that comprises complementary determining region (CDR) V_{H}CDR1, V_{H}CDR2, and V_{H}CDR3 sequences set forth in SEQ ID NOs: 1-3, respectively, and a light chain variable region (V_{L}) sequence that comprises complementary determining region V_{L}CDR1, V_{L}CDR2, and V_{L}CDR3 sequences set forth in SEQ ID NOs: 4-6, respectively; or
a V_{H} sequence that comprises V_{H}CDR1, V_{H}CDR2, and V_{H}CDR3 sequences set forth in SEQ ID NOs: 7-9, respectively, and V_{L} sequence that comprises V_{L}CDR1, V_{L}CDR2, and V_{L}CDR3 sequences set forth in SEQ ID NOs: 10-12, respectively.

55. The patient care kit of claim 54, wherein:
the V_{H} sequence comprises a sequence at least 80, 85, 90, 95, 97, 98, 99, or 100% identical to SEQ ID NO: 13, and the V_{L} sequence comprises a sequence at least 80, 85, 90, 95, 97, 98, 99, or 100% identical to SEQ ID NO: 14;
the V_{H} sequence comprises a sequence at least 80, 85, 90, 95, 97, 98, 99, or 100% identical to SEQ ID NO: 15, and the V_{L} sequence comprises a sequence at least 80, 85, 90, 95, 97, 98, 99, or 100% identical to SEQ ID NO: 16; or
the V_{H} sequence comprises a sequence at least 80, 85, 90, 95, 97, 98, 99, or 100% identical to SEQ ID NO: 17, and the V_{L} sequence comprises a sequence at least 80, 85, 90, 95, 97, 98, 99, or 100% identical to SEQ ID NO: 18.

56. (canceled)

57. The patient care kit of claim 53, wherein the means of (a) include reagents for performing immunohistochemistry (IHC) on one or more NEPC markers.

58. The patient care kit of claim 57, wherein the one or more NEPC markers are selected from synaptophysin, chromogranin A (CgA), neuron-specific enolase (NSE), and CD56.

59.-71. (canceled)


SEQUENCE LISTING (24 sequences)

SEQ ID NO: 1 - protein, 10 aa, synthetic construct
GFNIKDYYVH

SEQ ID NO: 2 - protein, 17 aa, synthetic construct
RIDVEDDETKYAPKFQG

SEQ ID NO: 3 - protein, 12 aa, synthetic construct
PIYGAREAWFAY

SEQ ID NO: 4 - protein, 12 aa, synthetic construct
TASSSVSSSYLH

SEQ ID NO: 5 - protein, 7 aa, synthetic construct
RTSNLAS

SEQ ID NO: 6 - protein, 9 aa, synthetic construct
HQYYRSPPT

SEQ ID NO: 7 - protein, 10 aa, synthetic construct
GFSLSTYSIS

SEQ ID NO: 8 - protein, 16 aa, synthetic construct
IIGDAGGIIYATWAKS

SEQ ID NO: 9 - protein, 7 aa, synthetic construct
DGTAFDI

SEQ ID NO: 10 - protein, 11 aa, synthetic construct
QASQSIYSKLG

SEQ ID NO: 11 - protein, 7 aa, synthetic construct
RASTLAS

SEQ ID NO: 12 - protein, 12 aa, synthetic construct
QQDYSYINVDNI

SEQ ID NO: 13 - protein, 121 aa, synthetic construct
EVQLVQSGAEVKKPGATVKISCKVSGFNIKDYYVHWVQQAPGKGLEWMGRIDVEDDETKYAPKFQGRVTITADTSTDTAYMELSSLRSEDTAVYYCATPIYGAREAWFAYWGQGTLVTVSS

SEQ ID NO: 14 - protein, 108 aa, synthetic construct
DIQMTQSPSSLSASVGDRVTITCTASSSVSSSYLHWYQQKPGKAPKLLIYRTSNLASGVPSRFSGSGSGTDFTLTISSLQPEDFATYYCHQYYRSPPTFGGGTKVEIK

SEQ ID NO: 15 - protein, 114 aa, synthetic construct
QSVKESEGGLFKPTDTLTLTCTVSGFSLSTYSISWVRQAPGNGLEWIGIIGDAGGIIYATWAKSRSTITRSTALNTVTLKMTGLTAADTATYFCARDGTAFDIWGPGTLVTVSS

SEQ ID NO: 16 - protein, 110 aa, synthetic construct
AYDMTQTPASVEVVVGGTVTIKCQASQSIYSKLGWYQQKPGQPPKLLIYRASTLASGVSSRFKGSGSGTEYTLTISGVQCDDAATYYCQQDYSYINVDNIFGGGTEVVVK

SEQ ID NO: 17 - protein, 114 aa, synthetic construct; identical to SEQ ID NO: 15

SEQ ID NO: 18 - protein, 110 aa, synthetic construct
AYDMTQTPASVEVVVGGTVTIKCQASQSIYSKLGWYQQKPGQPPKLLIYRASTLASGVSSRFKGSGSGTEYTLTISGVQADDAATYYCQQDYSYINVDNIFGGGTEVVVK

SEQ ID NO: 19 - DNA, 20 bp, Homo sapiens
  source 1..20: mol_type = genomic DNA
gctcttactcaactagcaga

SEQ ID NO: 20 - DNA, 20 bp, Homo sapiens
  source 1..20: mol_type = genomic DNA
gtaccctgcaacccaaagca

SEQ ID NO: 21 - DNA, 20 bp, Homo sapiens
  source 1..20: mol_type = genomic DNA
actacccctcccaccagaac

SEQ ID NO: 22 - DNA, 20 bp, Homo sapiens
  source 1..20: mol_type = genomic DNA
tttccttgggagatttgctg

SEQ ID NO: 23 - DNA, 20 bp, Homo sapiens
  source 1..20: mol_type = genomic DNA
gaaatgatgtgtccccaacc

SEQ ID NO: 24 - DNA, 20 bp, Homo sapiens
  source 1..20: mol_type = genomic DNA
agagccggaacaagacagaa